(19) United States
(12) Patent Application Publication
(10) Pub. No.: US 2017/0067008 A1
(43) Pub. Date: Mar. 9, 2017

(54) MEDIUM RETAINING CHAMBER AND CHAMBER SYSTEM FOR GROWTH AND MICROSCOPIC OBSERVATIONS OF CULTURED CELLS

(71) Applicant: The Board of Trustees of The University Of Illinois, Urbana, IL (US)

(72) Inventor: Lech Adam Kiedrowski, Lake Forest, IL (US)

(21) Appl. No.: 15/249,686

(22) Filed: Aug. 29, 2016

Related U.S. Application Data
(60) Provisional application No. 62/213,917, filed on Sep. 3, 2015.

(51) Int. Cl. C12M 1/34; C12M 1/00

(52) U.S. Cl. CPC C12M 41/46; C12M 23/26; C12M 23/22

(57) ABSTRACT

A chamber for a cell culture and a chamber holder system are disclosed. A representative chamber embodiment includes a first layer; and a second layer coupled to the first layer, the second layer further comprising a well having at least one side wall, the well extending through the second layer, wherein a predetermined portion of the first layer is substantially optically transmissive and is exposed in and forms a lower side of the well. The well may have a laminar flow shape, and may also include a plurality of recesses to accommodate the tips of inflow and outflow devices, such as for superfusion applications. The second layer may be comprised of a hydrophobic material or comprised of a material having a lower density than the density of culture medium to provide a buoyant chamber for sandwich cell cultures, along with submersible chambers.

### CROSS-REFERENCE TO A RELATED APPLICATION

This application is a nonprovisional of and claims the benefit of and priority to U.S. Provisional Patent Application No. 62/213,917, filed Sep. 3, 2015, inventor Lech Kiedrowski, titled “Floating Upside-Down, Superfusion Ready Chamber to Grow and Image Cell Cultures”, which is commonly assigned herewith, and all of which is hereby incorporated herein by reference in its entirety with the same full force and effect as if set forth in its entirety herein.

### STATEMENT REGARDING FEDERALLY SPONSORED RESEARCH OR DEVELOPMENT

This invention was made with government support under grant No. 5R21NS082786-02 awarded by the National Institutes of Health. The government has certain rights in this invention.

### FIELD OF THE INVENTION

The present invention, in general, relates to a cell culture chamber and a cell chamber holder, and more specifically, relates to a cell culture chamber that can be used in cell culturing, superfusion, and biological imaging.

### BACKGROUND OF THE INVENTION

As the biotechnology, agricultural, and medical fields have advanced, there has been an increasing need for growing cells in culture. The growth of cells in culture provides a controlled setting for the growth of sensitive cells, genetically transformed cells, or other cells which may require careful attention and/or a controlled environment. Various aspects of neuronal physiology and disease can be studied using cultured cells. For example, to study changes in intracellular pH or fluxes of ions such as Ca²⁺, Zn²⁺, or Na⁺, the cells can be loaded with ion-specific fluorescent probes or can be transfected with genetically encoded fluorescent ion sensors.

Most commonly, cells are cultured in plastic Petri dishes filled with media that support cell growth. Due to the optics and thickness of most Petri dishes, however, the cultured cells cannot be imaged properly while retained in the Petri dish.

For example, these dishes are not appropriate for experiments that employ fluorescent probes, because the plastic typically used to manufacture the dishes (polystyrene) is itself fluorescent. Moreover, the cells grown in plastic Petri dishes cannot be viewed using powerful objectives with a high numerical aperture because these objectives have a working distance that is too short to focus on the cells via the thick (about 0.5-0.8 mm) plastic on which the cells grow. Such plastic also is not compatible with objectives using differential interference contrast (DIC) that is important to study morphological features of the cells. To overcome these problems, researchers have plated cell cultures on glass coverslips which are thin, nonfluorescent, and DIC-compatible.

In order to view the cultured cells or to perform various experiments, the coverslips having the growing cell cultures are removed from the culture medium and the Petri dish, and placed in dedicated imaging chambers. These maneuvers, however, are associated with a stress to the cells caused by a direct exposure of the cells to air. While some cells may recover from this exposure, others may not, and may continue to exhibit corresponding detrimental effects, such as an unwanted release of calcium ions from neurons, which may interfere with the experimental measurements.

For microscopic observations of cultured neurons, low density cultures are also preferred because single neurons can be studied without the distraction of other cells in the background. To this end, “sandwich” neuronal-glial co-cultures have been developed, in which a glial feeder layer has been plated in advance on the bottom of a Petri dish, and neurons are grown on the underside of a glass coverslip, positioned to be above and facing the lower glial feeder layer. The neurons then benefit from the trophic factors released from the glia, and since a cytostatic agent is added to prevent glial proliferation, virtually only neurons grow on the coverslips.

In these sandwich co-cultures, however, to avoid mechanical damage to the different types of cells, there should be a small spacing or other gap between the coverslip with neurons and the glial feeder layer. To create this gap and keep the different types of cells spaced apart from each other, in the prior art, the underside of a coverslip is equipped with protruding “feet”, typically made of a paraffin wax. These feet are difficult to manufacture. More problematic, however, the feet need to be removed before placing the coverslip in a holder designed to work with a microscope, and once the coverslip is removed from the dish, the cultured cells are also subject to cytotoxic exposure to air. Because the cells are exposed to air during the time required to perform these maneuvers (e.g., greater than one second), cytotoxic types of stresses occur to the cells being studied and interfere with the desired experiments.

Perfusion also can require significant amounts of fluid to be used to bathe the cells being studied. Temperature control can be difficult, as the temperature of the entire chamber must be controlled.

Accordingly, a need remains for a versatile cell culturing chamber and chamber system which provides for the cells being cultured to remain submerged in a cell culture medium and avoid exposure to air, particularly when being imaged or transported out of a Petri dish for superfusion and other experiments, treatments, or imaging. Such a cell culturing chamber and chamber system should also provide for the capability to grow sandwich co-cultures, without modification of the apparatus and system, such as without the need to add wax feet to separate the co-cultured cells. Such a cell culturing chamber and chamber system should also be superfusion-ready, to facilitate superfusion of the cell cultures directly within the apparatus and system, and further, should not interfere with fluorescent probes and should be DIC-compatible with high numerical aperture objectives.

### SUMMARY OF THE INVENTION

The representative embodiments of the present invention provide numerous advantages. As mentioned above, representative embodiments provide a cell culturing chamber, as an article of manufacture, and a chamber holder system, typically used for holding the chamber during imaging of the growing cells. The representative cell culturing chamber embodiments include one or more wells which function to contain the cell culture medium over the growing cells, both when growing within a Petri dish and when being removed from and transported away from the Petri dish. This novel cell culturing chamber structure provides for the cells being cultured to always remain submerged in a cell culture medium and avoid exposure to air, particularly when being imaged or transported out of a Petri dish for superfusion and other experiments, treatments, or imaging.

The representative cell culturing chamber embodiments also provide for the capability to grow sandwich co-cultures, without modification of the chamber and holder system, and provide for both floating and fully submerged cell culturing chambers. The representative cell culturing chamber embodiments are also superfusion-ready, to facilitate superfusion of the cell cultures directly within the cell culturing chamber and chamber system.

In addition, as discussed in greater detail below, the wells (recesses or holes) of the cell culturing chamber may also be selectively sized and spaced to allow for appropriate cell growth while simultaneously diminishing or minimizing the required amounts of reagents used in experiments, many of which are quite costly, such as various antibodies, fluorescent markers or other reagents used in immunochemistry, for example and without limitation. Multiple wells may also be implemented in the same cell culturing chamber. The wells may have any selected shape, such as circular, square, rectangular, or may be shaped to provide for laminar flow during superfusion, such as oval (elliptical) or rhomboid, for example and without limitation. Various indicia or other markings, detents and recesses may be provided for orientation of the cell culturing chamber, along with microscopic grids, useful for imaging and for performing experiments on the cell cultures.

A representative chamber for cell culture may include a first layer secured to a second layer to form a laminate structure, while in other representative embodiments, the structures of these layers are combined to form a non-laminate, singular structure. The second layer and first layer define a well, as a contained volume enclosed on the sides by one or more walls of the second layer surrounding the well and enclosed on the bottom by the portion of the first layer beneath and/or adjacent the well (or hole). The well, as an option, may have at one or more recesses or cutouts. The at least one recess or cutout may be configured to accommodate the tip of an aspiration tube or an influx tube. In a case where there are at least two cutouts, one may be configured to accommodate the tip of an aspiration tube and one may be configured to accommodate the tip of an influx tube. The well may have a capacity within the range of 10 to 50 microliters, for example.

The representative chamber may be comparatively low density or lightweight. At least one component of the chamber material may be hydrophobic. This may allow the representative chamber to float when placed in a liquid such as a cell culture medium, particularly when the representative chamber is placed in a generally horizontal position into the liquid. In a representative embodiment, the chamber material may include at least one of a second layer of polystyrene and a first layer of glass. Floating chambers also may enable the effective growth and use of small amounts of neurons in mini-cultures.

A representative embodiment of a chamber for a cell culture is disclosed, the chamber insertable into a dish having a culture medium, with the representative chamber embodiment comprising: a first layer; and a second layer coupled to the first layer to form a laminate structure, the second layer further comprising a well having at least one side wall, the well extending through the second layer, the well further having a first end and a second end, the well further having a laminar flow shape extending laterally between the first and second ends; and wherein a predetermined portion of the first layer is substantially optically transmissive and is exposed in and forms a lower side of the well.

In a representative embodiment, the predetermined portion of the first layer may further comprise a micro-grid. In a representative embodiment, the second layer may further comprise at least one alignment recess, alignment detent, or alignment indicia.

In a representative embodiment, the second layer may further comprise a plurality of recesses in the at least one side wall, a first recess of the plurality of recesses arranged at the first end and having a size adapted to accommodate a tip of an inflow device, and a second recess of the plurality of recesses spaced apart from and arranged at the second end opposite the first recess, the second recess having a size adapted to accommodate a tip of an outflow device.

In a representative embodiment, the well may have a volume with a range of 10 to 50 microliters. In a representative embodiment, the laminar flow shape may be selected from the group consisting of: elliptical, oval, parallelogram, rhombus, rhomboid, and combinations thereof.

In another representative embodiment, the second layer may further comprise: a plurality of wells, the plurality of wells are coplanar with each other, each well having at least one side wall, each well extending through the second layer, and wherein a plurality of predetermined portions of the first layer are substantially optically transmissive and are exposed in and form corresponding lower sides of the plurality of wells. In such a representative embodiment, the second layer may further comprise: at least one channel coupling a first well of the plurality of wells to a second well of the plurality of wells.

In another representative embodiment, the second layer may further comprise a rim, the rim arranged around the circumference of the second layer.

In a representative embodiment, the second layer may be comprised of a hydrophobic material or may be comprised of a material having a first density less than a second density of a selected culture medium to provide that the chamber is buoyant in a selected culture medium. For example, in a representative embodiment, second layer comprises a biocompatible or inert closed-cell polymeric foam.

In a representative embodiment, the well has a size adapted to retain culture medium within the well independently of a position of the chamber.

In another representative embodiment, the second layer further comprises a groove recessed into an upper surface of the second layer, and further comprising an annular chamber holder system, the annular chamber holder system comprising: a first holder; a flexible gasket adapted to secure and seal the chamber against the first holder, wherein the flexible gasket further comprises a raised ring to mate with the groove of the second layer.

Another representative embodiment of a chamber for a cell culture is disclosed, the chamber insertable into a dish having a selected culture medium, the chamber buoyant in the selected culture medium, the chamber comprising: a first layer comprising a substantially optically transmissive material; and a second layer coupled to the first layer to form a laminate structure, the second layer further comprising a well having at least one side wall, the well extending through the second layer; wherein a predetermined portion of the first layer is substantially optically transmissive and is exposed in and forms a lower side of the well; and wherein the second layer is comprised of a hydrophobic material or is comprised of a material having a first density less than a second density of the selected culture medium.

In such a representative embodiment, for example, the second layer comprises a biocompatible or inert closed-cell polymeric foam.

In such a representative embodiment, the well may further comprise a first end and a second end, with the well further having a laminar flow shape extending laterally between the first and second ends.

In such a representative embodiment, the second layer may further comprise a plurality of recesses in the at least one side wall, a first recess of the plurality of recesses arranged at the first end and having a size adapted to accommodate a tip of an inflow device, and a second recess of the plurality of recesses spaced apart from and arranged at the second end opposite the first recess, the second recess having a size adapted to accommodate a tip of an outflow device. In such a representative embodiment, the laminar flow shape may be selected from the group consisting of: elliptical, oval, parallelogram, rhombus, rhomboid, and combinations thereof.

In such a representative embodiment, the well may have a size adapted to retain culture medium within the well independently of a position of the chamber.

In a representative embodiment, the second layer comprises a biocompatible or inert polymer or copolymer, selected from the group consisting of: fluorinated polymers or copolymers including poly(vinylidene fluoride), poly(vinylidene fluoride-co-hexafluoropropene), poly(tetrafluoroethylene), expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-co-trimethylene carbonate); poly(phosphoesters); poly(phosphoester urethane); poly(trimethylene carbonate); poly(iminocarbonate); poly(ethylene); poly(propylene) co-poly(ether-esters), including poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers including poly(vinyl chloride); poly(vinyl ethers) including poly(vinyl methyl ether); poly(vinylidene halides) including poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins including poly(ethylene-co-vinyl alcohol) (EVAL), copolymers of acrylonitrile-styrene, ABS resins, and copolymers of ethylene-vinyl acetate; poly(amides); poly(caprolactam); alkyd resins; poly(carbonates); poly(oxymethylenes); poly(imides); poly(ester amides); poly(ethers) including poly(alkylene glycols) including poly(ethylene glycol) and poly(propylene glycol); a poly(ester amide), a poly(lactide) or a poly(lactide-co-glycolide) copolymer; epoxy resins; polyurethanes; rayon; rayon-triacetate; biomolecules including fibrin, fibrinogen, starch, poly(amino acids); peptides, proteins, gelatin, chondroitin sulfate, dermatan sulfate (a copolymer of D-glucuronic acid or L-iduronic acid and N-acetyl-Dgalactosamine), collagen, hyaluronic acid, glycosaminoglycans; polysaccharides including poly(N-acetylglucosamine), chitin, chitosan, cellulose, cellulose acetate, cellulose butyrate, cellulose acetate butyrate, cellophane, cellulose nitrate, cellulose propionate, cellulose ethers, carboxymethylcellulose; and any derivatives, analogs, homologues, congeners, salts, copolymers and combinations thereof.

A plurality of chambers for sandwich cell cultures are disclosed, the plurality of chambers insertable into a dish having a selected culture medium, with the plurality of chambers comprising: a first chamber; and a second chamber; each of the first and second chambers comprising: a first layer; and a second layer coupled to the first layer to form a laminate structure, the second layer further comprising a well having at least one side wall, the well extending through the second layer, and wherein a predetermined portion of the first layer is substantially optically transmissive and is exposed in and forms a lower side of the well; wherein the second layer of the first chamber is comprised of a hydrophobic material or is comprised of a material having a first density less than a second density of the selected culture medium to provide that the first chamber is buoyant in the selected culture medium; and wherein the second layer of the second chamber is comprised of a material having a third density greater than the second density of the selected culture medium to provide that the second chamber is submergible in the selected culture medium.

In such a representative embodiment, at least one well may further comprise a first end and a second end, the at least one well further having a laminar flow shape extending laterally between the first and second ends, wherein the laminar flow to shape is selected from the group consisting of: elliptical, oval, parallelogram, rhombus, rhomboid, and combinations thereof.

In another representative embodiment, a plurality of chambers for sandwich cell cultures are also disclosed, the plurality of chambers comprising: a first and a second chamber, each of the first and second chambers comprising: a first layer; a second layer coupled to the first layer, the second layer further comprising a well having at least one side wall, the well extending through the second layer, and wherein a predetermined portion of the first layer is exposed in and forms a lower side of the well; wherein the first chamber is buoyant in a culture medium and arranged upside down; and wherein the second chamber is submergible in the culture medium and arranged upside up underneath the first chamber.

Another representative embodiment of a chamber for a cell culture is disclosed, the chamber comprising: a first layer; and a second layer coupled to the first layer to form a laminate structure, the second layer further comprising a well having at least one side wall, the well extending through the second layer, and wherein a predetermined portion of the first layer is exposed in and forms a lower side of the well.

Another representative embodiment of a chamber for a cell culture is disclosed, the chamber insertable into a dish having a culture medium, the chamber comprising: a first layer; and a second layer coupled to the first layer to form a laminate structure, the second layer further comprising a well having at least one side wall, the well extending through the second layer, and wherein a predetermined portion of the first layer is exposed in and forms a lower side of the well.

In such a representative embodiment, the predetermined portion of the first layer may be substantially optically transmissive. In a representative embodiment, the predetermined portion of the first layer may further comprise a micro-grid. In a representative embodiment, the second layer may further comprise at least one alignment recess, alignment detent, or alignment indicia. In a representative embodiment, the second layer may further comprise a rim, the rim arranged around the circumference of the second layer.

In a representative embodiment, the second layer may be coupled to the first layer with an adhesive.

In a representative embodiment, the second layer may further comprise at least one recess in the at least one side wall, the at least one recess having a size adapted to accommodate a tip of an inflow or outflow device.

In another representative embodiment, the second layer may further comprise a plurality of recesses in the at least one side wall, a first recess of the plurality of recesses having a size adapted to accommodate a tip of an inflow device, and a second recess of the plurality of recesses spaced apart from and arranged opposite the first recess, the second recess having a size adapted to accommodate a tip of an outflow device.

In a representative embodiment, the second layer may further comprise a plurality of wells, the plurality of wells are coplanar with each other, each well having at least one side wall, each well extending through the second layer, and wherein a plurality of predetermined portions of the first layer are exposed in and form corresponding lower sides of the plurality of wells. In such a representative embodiment, the second layer may further comprise at least one channel coupling a first well of the plurality of wells to a second well of the plurality of wells.

In a representative embodiment, the first layer may comprise a substantially optically transmissive borosilicate glass or polystyrene latex.

Another representative embodiment of a chamber for a cell culture is disclosed, the chamber comprising: a first layer comprising a substantially optically transmissive material; and a second layer coupled to the first layer to form a laminate structure, the second layer further comprising a well having at least one side wall, the well extending through the second layer, the second layer further comprising a first recess and a second recess in the at least one side wall, the second recess spaced apart from and arranged opposite the first recess, and wherein a predetermined portion of the first layer is exposed in and forms a lower side of the well.

Another representative embodiment of a chamber for a cell culture is disclosed, the chamber insertable into a dish having a selected culture medium, the chamber comprising: a first layer comprising a substantially optically transmissive material; and a second layer coupled to the first layer to form a laminate structure, the second layer further comprising a well having at least one side wall, the well extending through the second layer; wherein a predetermined portion of the first layer is exposed in and forms a lower side of the well; and wherein the second layer is comprised of a material having a first density less than a second density of the selected culture medium to provide that the chamber is buoyant in a selected culture medium.

Numerous other advantages and features of the present invention will become readily apparent from the following detailed description of the invention and the embodiments thereof, from the claims and from the accompanying drawings.

### BRIEF DESCRIPTION OF THE DRAWINGS

The objects, features and advantages of the present invention will be more readily appreciated upon reference to the following disclosure when considered in conjunction with the accompanying drawings, wherein like reference numerals are used to identify identical components in the various views, and wherein reference numerals with alphabetic characters are utilized to identify additional types, instantiations or variations of a selected component embodiment in the various views, in which:

Figure (or “FIG.”) 1 is an isometric view of a representative first chamber embodiment.

Figure (or “FIG.”) 2 is a cross-sectional view of the representative first chamber embodiment illustrated in FIG. 1.

Figure (or “FIG.”) 3 is an isometric view of the representative components and a representative method of assembly of a representative first chamber embodiment.

Figure (or “FIG.”) 4 is an isometric view of the representative first chamber embodiment arranged in a Petri dish with a cell culture and a culture medium.

Figure (or “FIG.”) 5 is a cross-sectional view of the representative first chamber embodiment arranged in a Petri dish illustrated in FIG. 4.

Figure (or “FIG.”) 6 is a cross-sectional view showing in greater detail the well region of the representative first chamber embodiment arranged in the Petri dish illustrated in FIG. 4.

Figure (or “FIG.”) 7 is an isometric view of two representative first chamber embodiments arranged in a cell culture sandwich configuration in a Petri dish.

Figure (or “FIG.”) 8 is a cross-sectional view of the two representative first chamber embodiments arranged in a cell culture sandwich configuration in a Petri dish illustrated in FIG. 7.

Figure (or “FIG.”) 9 is an isometric view of a representative second chamber embodiment.

Figure (or “FIG.”) 10 is an isometric view of a representative third chamber embodiment.

Figure (or “FIG.”) 11 is a cross-sectional view of the representative second and/or third chamber embodiments illustrated in FIGS. 9 and 10, further arranged with influx and aspiration tubes for superfusion of cell cultures.

Figure (or “FIG.”) 12 is an isometric view of a representative fourth chamber embodiment.

Figure (or “FIG.”) 13 is a cross-sectional view of the representative fourth chamber embodiment illustrated in FIG. 12.

Figure (or “FIG.”) 14 is an isometric view of a representative fifth chamber embodiment.

Figure (or “FIG.”) 15 is a cross-sectional view of the representative fifth chamber embodiment illustrated in FIG. 14.

Figure (or “FIG.”) 16 is an isometric view of a representative sixth chamber embodiment having a micro-grid.

Figure (or “FIG.”) 17 is a cross-sectional view of the representative sixth chamber embodiment illustrated in FIG. 16.

Figure (or “FIG.”) 18 is an isometric view of a representative seventh chamber embodiment showing representative alignment detents, recesses, and markings, for use in any representative chamber embodiment.

Figure (or “FIG.”) 19 is an isometric view of a representative eighth chamber embodiment.

Figure (or “FIG.”) 20 is a cross-sectional view of the representative eighth chamber embodiment illustrated in FIG. 19.

Figure (or “FIG.”) 21 is an isometric view of a representative first chamber holder system embodiment.

Figure (or “FIG.”) 22 is a cross-sectional view of the representative first chamber holder system embodiment illustrated in FIG. 21.

Figure (or “FIG.”) 23 is an isometric view of a representative second chamber holder system embodiment.

Figure (or “FIG.”) 24 is a cross-sectional view of the representative second chamber holder system embodiment illustrated in FIG. 23.

### DETAILED DESCRIPTION OF REPRESENTATIVE EMBODIMENTS

While the present invention is susceptible of embodiment in many different forms, there are shown in the drawings and will be described herein in detail specific exemplary embodiments thereof, with the understanding that the present disclosure is to be considered as an exemplification of the principles of the invention and is not intended to limit the invention to the specific embodiments illustrated. In this respect, before explaining at least one embodiment consistent with the present invention in detail, it is to be understood that the invention is not limited in its application to the details of construction and to the arrangements of components set forth above and below, illustrated in the drawings, or as described in the examples. Methods and apparatuses consistent with the present invention are capable of other embodiments and of being practiced and carried out in various ways. Also, it is to be understood that the phraseology and terminology employed herein, as well as the abstract included below, are for the purposes of description and should not be regarded as limiting.

As mentioned above, representative embodiments provide a cell culturing chamber (100, 200, 300, 400, 500, 600, 700, 800), as an article of manufacture, and a chamber holder system (250, 350), typically used for holding the cell culturing chamber (100, 200, 300, 400, 500, 600, 700, 800) during imaging of the growing cells. The representative cell culturing chamber embodiments (100, 200, 300, 400, 500, 600, 700, 800) are insertable into a dish having a removable cover, such as a Petri dish 120, and a cell culture medium.

The representative cell culturing chamber embodiments (100, 200, 300, 400, 500, 600, 700, 800) include one or more wells 150 which function to contain the cell culture medium over the growing cells, both when growing within a Petri dish and when being removed from and transported away from the Petri dish, providing for the cells being cultured to always remain submerged in a cell culture medium and avoid exposure to air, particularly when being imaged or transported out of a Petri dish for superfusion and other experiments, treatments, or imaging. The representative cell culturing chamber embodiments (100, 200, 300, 400, 500, 600, 700, 800) also provide for the capability to grow sandwich co-cultures, without modification of the chamber and holder system, and provide for both floating and fully submerged cell culturing chambers. The representative cell culturing chamber embodiments (100, 200, 300, 400, 500, 600, 700, 800) are also superfusion-ready, to facilitate superfusion of the cell cultures directly within the cell culturing chamber and chamber system.

In addition, as discussed in greater detail below, the wells 150 (recesses or holes) of the cell culturing chamber (100, 200, 300, 400, 500, 600, 700, 800) may also be selectively sized and spaced to allow for appropriate cell growth while simultaneously diminishing or minimizing the required amounts of reagents used in experiments, many of which are quite costly, such as various antibodies, fluorescent markers or other reagents used in immunochemistry, for example and without limitation. Multiple wells 150 may also be implemented in the same cell culturing chamber. The wells 150 may have any selected shape, such as circular, square, rectangular, or may be shaped to provide for laminar flow during superfusion, such as oval (elliptical) or rhomboid, for example and without limitation. Various markings, detents and recesses may be provided for orientation of the cell culturing chamber, along with microscopic grids, useful for imaging and for performing experiments on the cell cultures.

A representative chamber (100, 200, 300, 400, 500, 600, 700, 800) that requires only a small amount of liquid to maintain cells submerged in a culture medium is disclosed. Cells may be cultured directly in the chamber (100, 200, 300, 400, 500, 600, 700, 800). The representative chamber (100, 200, 300, 400, 500, 600, 700, 800) may be configured so that superfusion can take place directly within the chamber (100, 200, 300, 400, 500, 600, 700, 800) without the need to move the culture to another chamber. Thus, once the cells have been cultured, there is no need to transfer the cells to other chambers to conduct superfusion. The cells grown in the chambers (100, 200, 300, 400, 500, 600, 700, 800) detailed herein may be continuously submerged in culture medium, and may never be directly exposed to air. The representative chamber may be designed and sized to culture small quantities of neurons. As such, a representative chamber (100, 200, 300, 400, 500, 600, 700, 800) may contain a comparatively small well 150, with a capacity of about 10 to 50 microliters, for example and without limitation.

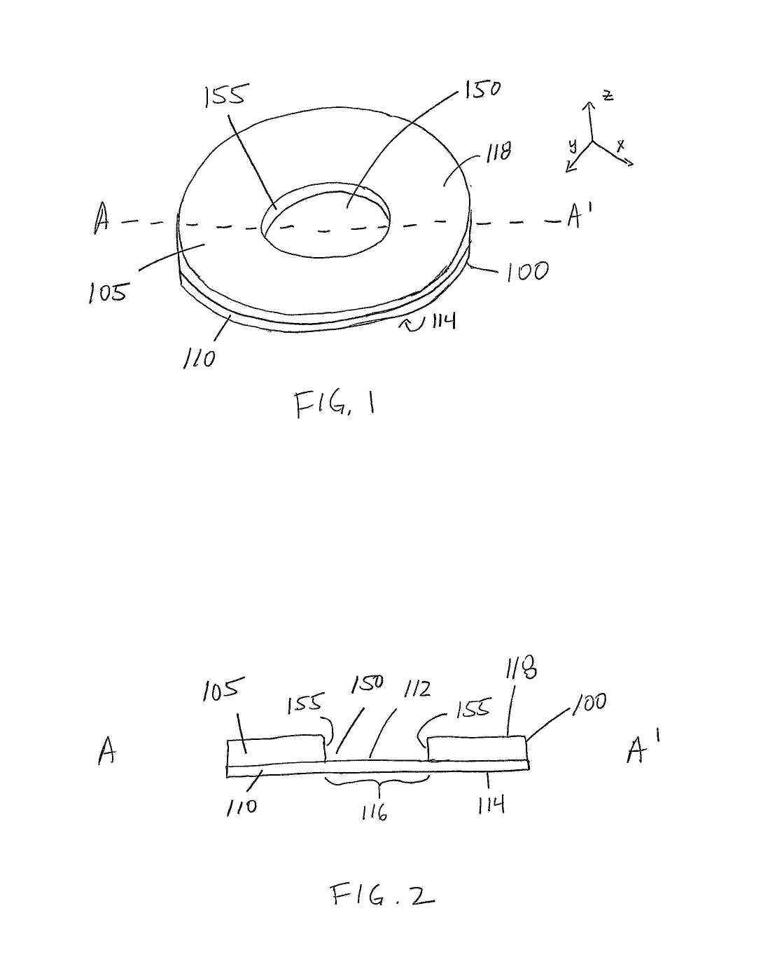

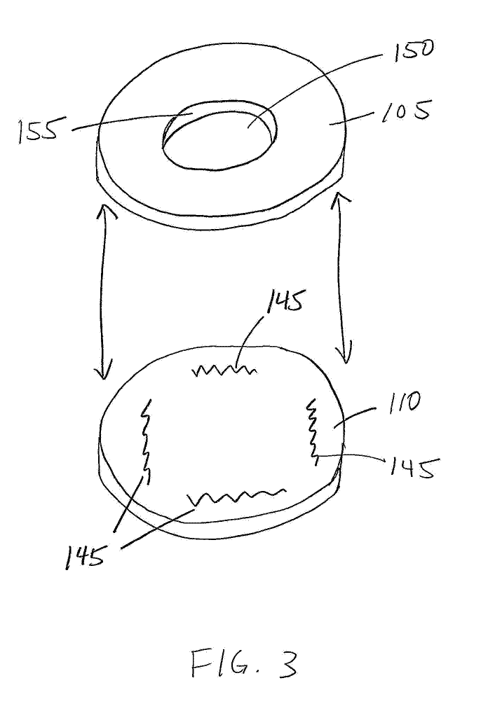

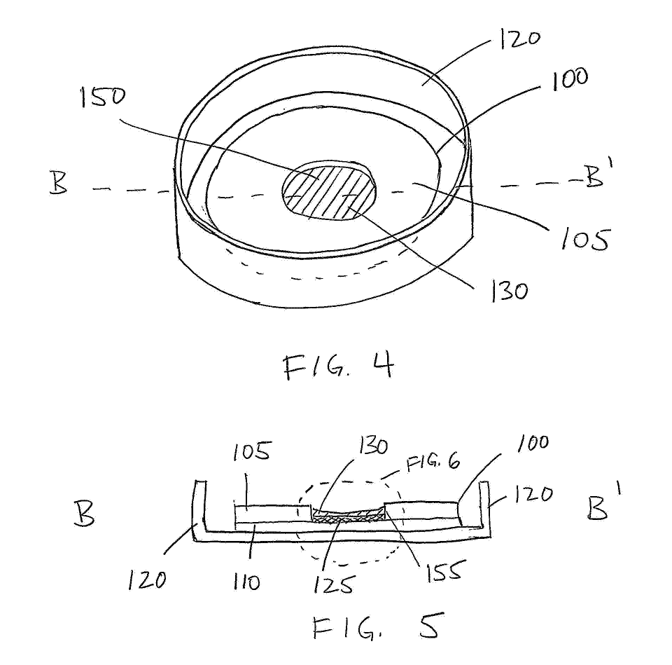

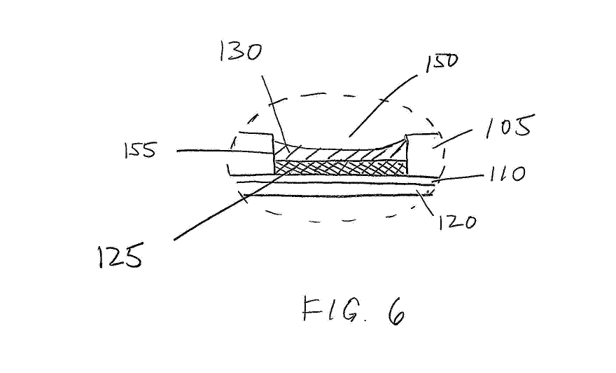

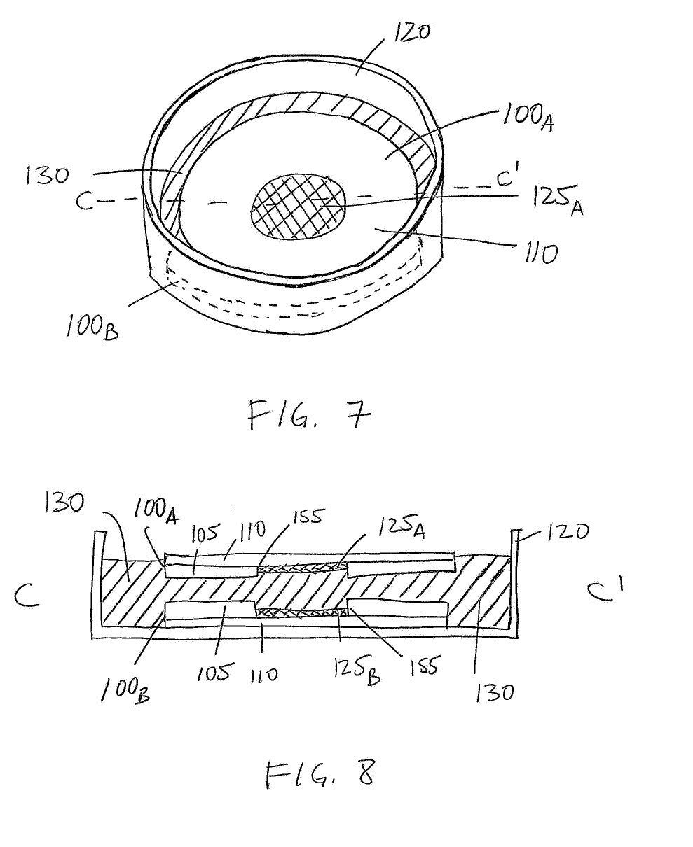

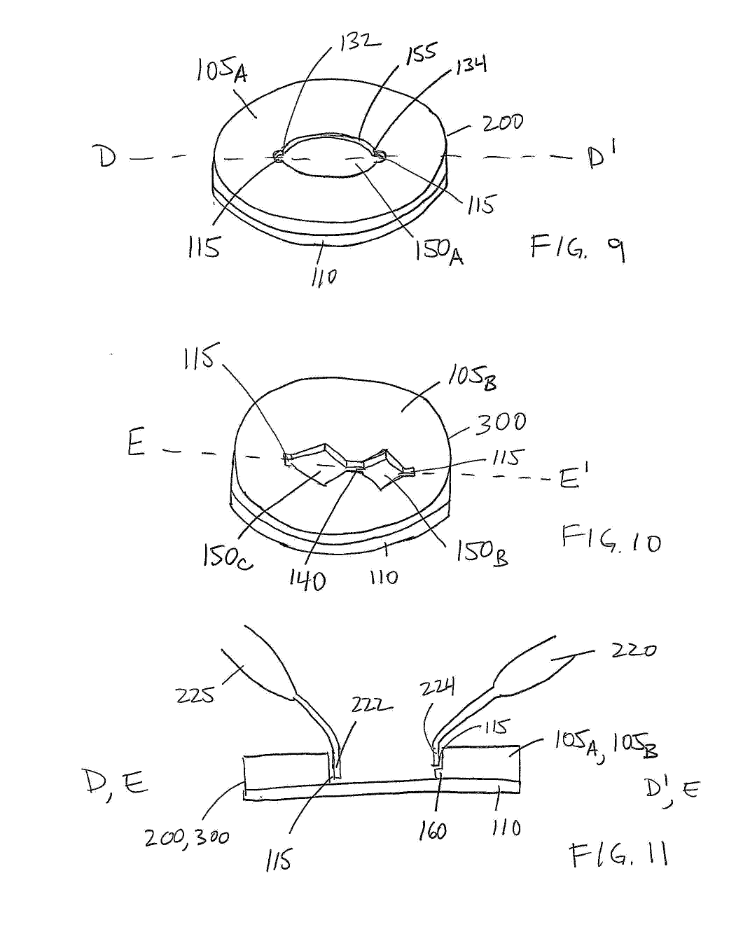

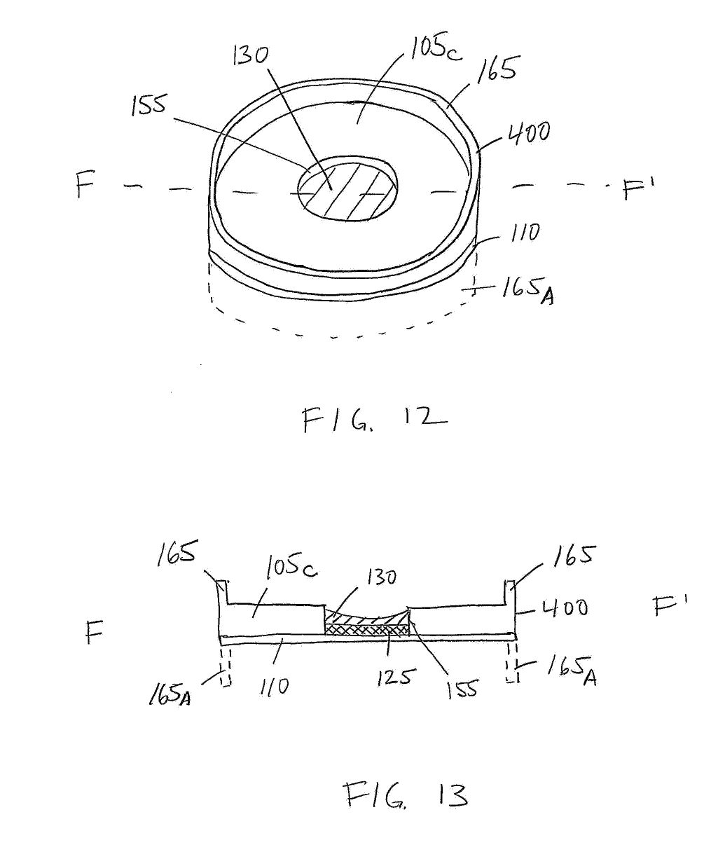

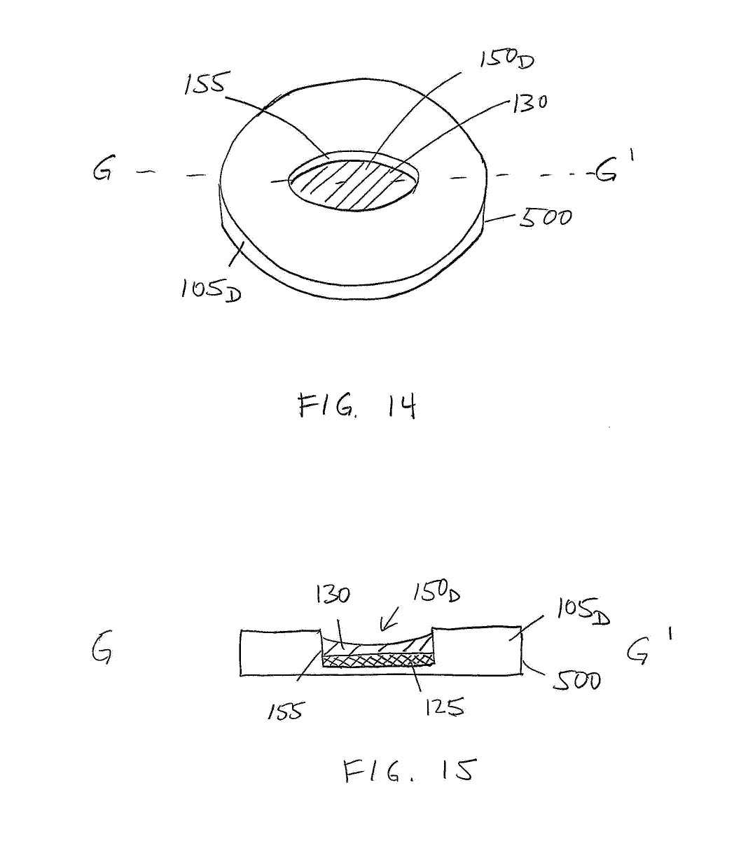

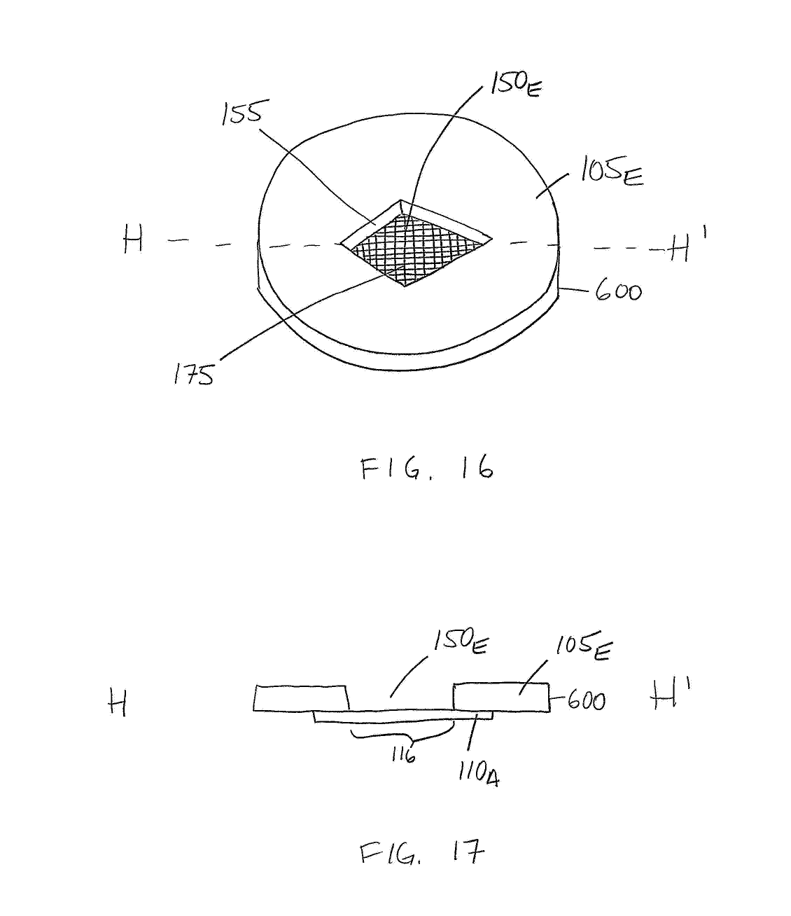

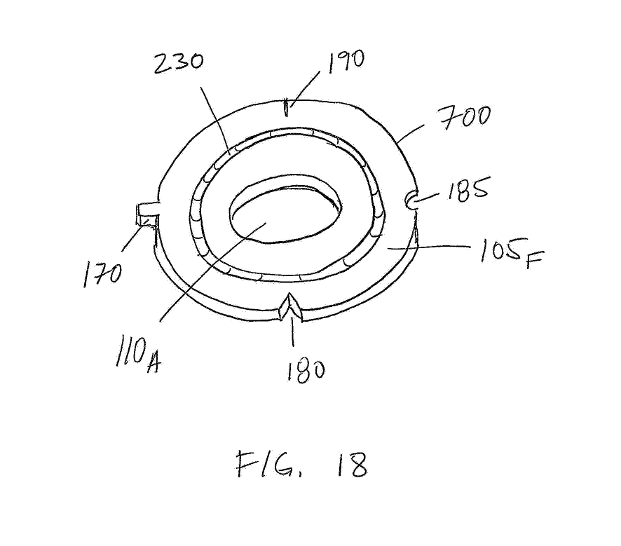

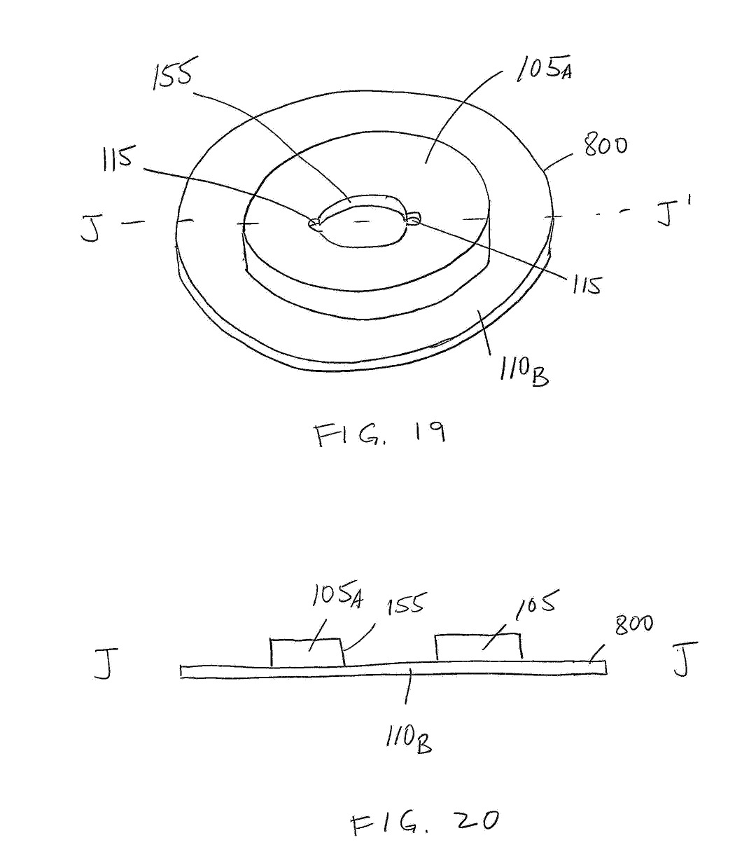

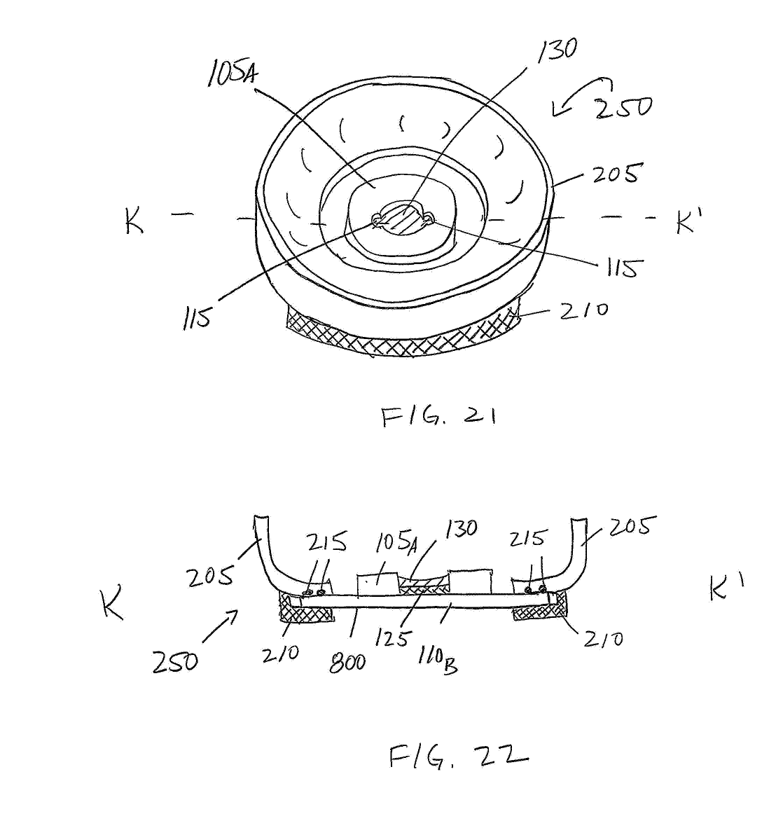

FIG. 1 is an isometric view of a representative first chamber 100 embodiment. FIG. 2 is a cross-sectional view (through the A-A′ plane) of the representative first chamber 100 embodiment illustrated in FIG. 1. FIG. 3 is an isometric view of the representative components and a representative method of assembly of a representative first chamber embodiment. FIG. 4 is an isometric view of the representative first chamber 100 embodiment arranged in a Petri dish 120 with a cell culture 125 and a culture medium 130 covering the cell culture 125. FIG. 5 is a cross-sectional view (through the B-B′ plane) of the representative first chamber 100 embodiment arranged in a Petri dish 120 illustrated in FIG. 4. FIG. 6 is a cross-sectional view (also through the B-B′ plane) showing in greater detail the well 150 region of the representative first chamber 100 embodiment arranged in the Petri dish illustrated in FIG. 4.

Various representative chambers (100, 200, 300, 400, 600, 700, 800) generally are laminate structures comprising a first layer 110 coupled to a second layer 105, such as coupled by adhering the second layer 105 to the first layer 110 (or vice-versa) using an adhesive 145 as illustrated in FIG. 3, to form a representative chamber 100, as illustrated. A representative chamber (500) which is not a laminate structure, but is formed as a singular or unitary layer, however, is also within the scope of the disclosure and is discussed in greater detail below with reference to FIGS. 14 and 15. In addition, any of the second layer 105 or the first layer 110 may be comprised of or formed by component sublayers, of any suitable materials.

Continuing to refer to FIGS. 1-6, the second layer 105 and first layer 110 further comprise a well 150, which may be formed by a hole or other type of recess, typically a through hole extending the entire thickness or depth of the second layer 105 as illustrated, forming side walls 155 of the second layer 105, and with a portion (region 116) of the first layer 110 forming the bottom or lower side of the well 150. More specifically, within the region 116 of the first layer 110, a first side (or surface) 112 of the first layer 110 is exposed within the well 150, with the first layer 110 also forming the bottom or lower side of the well 150, as illustrated, with one or more walls 155 of the second layer 105 further defining the well 150. The first side (or surface) 112 of the first layer 110 which is within the well 150 of the chamber (100, 200, 300, 400, 500, 600, 700, 800) (region 116) is utilized for growth of a cell culture 125, which in turn is completely covered by a culture medium 130. While the well 150 is illustrated in several of the various Figures as being in the center of or otherwise centrally located within the chamber (100, 200, 300, 400, 500, 600, 700, 800), those having skill in the art will recognize that the well 150 may have any location within the chamber (100, 200, 300, 400, 500, 600, 700, 800), such as illustrated in FIG. 10.

Stated another way, the second layer 105 and first layer 110 define a well 150, as a contained volume enclosed on all sides by the wall(s) 155 of the second layer 105 surrounding the well 150, and enclosed on the bottom or lower side (or other orthogonal or perpendicular side) by the portion (region 116) of the first layer 110 beneath and/or adjacent the well (or hole) 150. In representative chamber embodiments (100, 200, 300, 400, 500, 600, 700, 800), the well 150 contains the cell culture 125 growing on the first side 112 of the first layer 110, and the cell culture 125 is covered completely by the culture medium 130.

The first side 118 of the chamber (100, 200, 300, 400, 500, 600, 700, 800) having the well 150 is then considered the upper side (or upside) of the chamber (100, 200, 300, 400, 500, 600, 700, 800), and the second side 114 of the chamber (100, 200, 300, 400, 500, 600, 700, 800) is then considered the lower (or down) side of the chamber (100, 200, 300, 400, 500, 600, 700, 800). As mentioned above, the representative chambers (100, 200, 300, 400, 500, 600, 700, 800) may be utilized in both arrangements, with the upside (118) facing upward (and the lower side 114 facing downward) or with the upside (118) facing downward (and the lower side 114 facing upward), as illustrated in FIGS. 7 and 8.

The second layer 105 may be opaque to light or may be substantially optically transmissive to light. The first layer 110, or more particularly the part of the first layer 110 which is exposed below or within the well 150 (region 116), is substantially optically transmissive for a selected wavelength range, such as substantially optically transmissive in the visible spectrum for imaging using a light microscope, or in the ultraviolet (uv) spectrum for imaging using other techniques, for example and without limitation. This allows the cell culture 125 to be directly imaged, such as through light microscopy, all while the cell culture 125 remains undisturbed within the well 150 and completely covered by the culture medium 130, avoiding any exposure of the cell culture 125 to air. For example, the first layer 110, or more particularly the part of the first layer 110 which is exposed below or within the well 150 (region 116), may be comprised of a glass, a film (e.g., a fluorocarbon film), or any other comparatively transparent polymer or plastic which can be fabricated to be thin enough (e.g., 0.1-0.2 mm, for example and without limitation) to allow focusing on the cell culture 125 using a microscope objective.

In representative embodiments, the well 150 is generally sized such that hydrostatic forces retain the culture medium 130 within the well 150. For example, given that enough culture medium 130 has been provided (e.g., at least a predetermined minimum amount), and depending upon the selected embodiment, even when the chamber (100, 200, 300, 400, 500, 600, 700, 800) is removed from the Petri dish, whether upside down, upside up or sideways, and whether static or moving, the culture medium 130 remains within the well 150 and is completely covering the cell culture 125. As mentioned above, the well 150 may have any shape and size, such as circular, triangular, square, rectangular, polygonal, or may be shaped to provide for laminar flow during superfusion, such as oval (ovoid or elliptical), rhomboid, or another shape suitable for allowing generally laminar flow. For example and without limitation, a well 150 used for superfusion studies may be shaped to promote laminar flow (e.g., elliptical or oval, rhomboid) and comparatively small (e.g., fitting within a circle having a diameter of 4-8 mm); a well 150 used for immunocytochemistry studies may have any shape (e.g., circular) and also comparatively small (e.g., a diameter of 4-8 mm); a well 150 used for the study of a larger number of cells may have any shape and be comparatively large (e.g., fitting within a circle having a diameter of 18-20 mm).

The representative chambers (100, 200, 300, 400, 500, 600, 700, 800) and/or the component second layer 105 and first layer 110, also may have any selected shape and size. The representative first chamber 100 and its component second layer 105 and first layer 110 are illustrated as substantially circular, generally to fit within a typical circular Petri dish 120, as illustrated in FIGS. 4 and 5. When both the second layer 105 and well 150 are substantially circular, as illustrated in FIGS. 1-6, the second layer 105 has an annular form or shape. For these embodiments, such as to fit within a typical 35 mm Petri dish 120 or existing holder systems, the outer diameter or outer lateral dimension of the representative chambers (100, 200, 300, 400, 500, 600, 700, 800) is typically less than or equal to 35 mm, in particular, an outer diameter between 15 and 35 millimeters, with the diameter of the well 150 ranging between 4 mm to 20 mm, e.g., 5 mm wide and 0.8-1.2 mm deep, all for example and without limitation.

For example and without limitation, the lateral dimensions (x-direction (x-dimension) and/or y-direction (y-dimension) illustrated in FIG. 1) of a representative chamber (100, 200, 300, 400, 500, 600, 700, 800) may be between 5 mm and 35 mm, 10 mm and 35 mm, 15 mm and 35 mm, 20 mm and 35 mm, 25 mm and 35 mm, and 30 mm and 35 mm, and so on. For example and without limitation, the lateral dimensions (x-direction and/or y-direction) of a representative well 150 may be between 1 mm and 30 mm, 3 mm and 30 mm, 5 mm and 30 mm, 7 mm and 30 mm, 10 mm and 30 mm, and 15 mm and 30 mm; 1 mm and 20 mm, 3 mm and 20 mm, 5 mm and 20 mm, 7 mm and 20 mm, 10 mm and 20 mm, and 15 mm and 20 mm; and so on. For example and without limitation, the depth of the well 150 (z-direction (z-dimension) illustrated in FIG. 1) may be between 0.1 mm-2 mm, 0.15 mm-2 mm, 0.2 mm-2 mm, 0.25 mm-2 mm, 0.3 mm-2 mm, 0.35 mm-2 mm, 0.4 mm-2 mm, 0.45 mm-2 mm, 0.5 mm-2 mm, 0.55 mm-2 mm, 0.6 mm-2 mm, 0.65 mm-2 mm, 0.7 mm-2 mm, 0.75 mm-2 mm, 0.8 mm-2 mm, 0.1 mm-1.5 mm, 0.15 mm-1.5 mm, 0.2 mm-1.5 mm, 0.25 mm-1.5 mm, 0.3 mm-1.5 mm, 0.35 mm-1.5 mm, 0.4 mm-1.5 mm, 0.45 mm-1.5 mm, 0.5 mm-1.5 mm, 0.55 mm-1.5 mm, 0.6 mm-1.5 mm, 0.65 mm-1.5 mm, 0.7 mm-1.5 mm, 0.75 mm-1.5 mm, 0.8 mm-1.5 mm, 0.1 mm-1.2 mm, 0.15 mm-1.2 mm, 0.2 mm-1.2 mm, 0.25 mm-1.2 mm, 0.3 mm-1.2 mm, 0.35 mm-1.2 mm, 0.4 mm-1.2 mm, 0.45 mm-1.2 mm, 0.5 mm-1.2 mm, 0.55 mm-1.2 mm, 0.6 mm-1.2 mm, 0.65 mm-1.2 mm, 0.7 mm-1.2 mm, 0.75 mm-1.2 mm, 0.8 mm-1.2 mm, and so on.

Alternatively, the representative chamber (100, 200, 300, 400, 500, 600, 700, 800) may be of another structure or shape such as triangular, square, rectangular, polygonal, ovoid, oval, elliptical, rhomboid, or other shape, having a maximum lateral dimension which is less than the largest diameter of any suitable Petri dish which will be utilized. Regardless of the structure or shape, the representative chamber (100, 200, 300, 400, 500, 600, 700, 800) may have any suitable dimensions that allow it to be placed within any selected container used for growing cell cultures, such as a standard 35 mm Petri dish (generally also having a separate cover, not separately illustrated), for example and without limitation.

For example and without limitation, in a representative embodiment, a representative chamber (100, 200, 300, 400, 500, 600, 700, 800) is generally circular and has a diameter of about 16 mm, and having a well 150 (e.g., a well 150_{A} or 150_{D}) which has an oval or elliptical shape in the lateral dimension, with a major axis of about 9 mm to 12 mm and a minor axis of about 3 mm to 7 mm.

The first layer 110 is comprised of any material that allows the growth of cells and is a substantially optically transmissive material for a selected range of imaging wavelengths, such as a biocompatible or inert glass or polymer, such as a borosilicate glass or polystyrene latex. The material of the first layer 110 may be compatible with differential interference contrast (DIC) and immersion lenses. In a representative embodiment, the first layer 110 may have a thickness between 0.08 mm to 0.19 mm, for example and without limitation. Also for example and without limitation, the first layer 110 may have the typical thickness and be comprised of the glass material of a standard glass cover slip used in the medical and biological sciences, whether square or circular or any other shape.

As described in greater detail below, the second layer 105 is comprised of any suitable material, such as a biocompatible or inert polymer or plastic, any type of biocompatible or inert glass, any type of biocompatible or inert polymeric foam, such as a closed cell foam, for example and without limitation. Any type of adhesive 145 (also generally biocompatible or inert) may be utilized to bond the second layer 105 and the first layer 110 to form a cell culturing chamber (100, 200, 300, 400, 500, 600, 700, 800), including without limitation silicone glues (for example SYLGARD®, 184 silicone elastomer kit), epoxies, cyanoacrylates, polyurethanes and other urethane and acrylic adhesives, polyimides.

The second layer 105 may have an outside perimeter that defines a shape, and the shape may be that of a circle (e.g., forming an annulus), or it may be in another shape such as triangular, square, rectangular, polygonal, ovoid, oval, elliptical, or other shape. The second layer 105 may define a well 150 or other hole, as shown in the Figures. The well 150 may be a circle, or it may be in another shape such as triangular, square, rectangular, polygonal, ovoid, oval, elliptical, or other shape. The second layer 105 may be comprised of plastic, such as polystyrene. The second layer 105 may have a diameter between 6 to 35 millimeters, in particular, between 8 and 31 mm. If the second layer 105 has a shape other than round, it may be dimensioned to allow it to fit within a 31 mm circle, for example and without limitation.

Referring again to FIG. 3, it should be noted that the representative chambers (100, 200, 300, 400, 600, 700, 800) may be fabricated using a wide range of techniques. For example and without limitation, the second layer 105 may be injection molded. In another embodiment, the second layer 105 is fabricated from a large sheet of polymeric material, a plurality of spaced-apart holes are formed in the large sheet (e.g., by cutting using a punch press), a plurality of first layers 110 are positioned (each over one of the holes) and adhered to the large sheet of second layers 105, and the representative chambers (100, 200, 300, 400, 600, 700, 800) are individuated or singulated (e.g., by cutting using a punch press). Those having skill in the art will recognize innumerable methods of manufacturing representative chambers (100, 200, 300, 400, 500, 600, 700, 800), any and all of which are considered equivalent and within the scope of the disclosure.

FIG. 7 is an isometric view of two representative first chamber embodiments 100_{A} and 100_{B} arranged in a cell culture sandwich configuration in a Petri dish 120 containing culture medium 130. FIG. 8 is a cross-sectional view (through the C-C′ plane) of the two representative first chamber embodiments arranged in a cell culture sandwich configuration in the Petri dish 120 illustrated in FIG. 7. As illustrated in FIGS. 7 and 8, the representative chambers (100, 200, 300, 400, 500, 600, 700, 800) may be utilized in both arrangements, with the upside (118) facing upward (and the lower side 114 facing downward), as illustrated for chamber 100_{B}, or with the upside (118) facing downward (and the lower side 114 facing upward), as illustrated for chamber 100_{A}.

As illustrated for chamber 100_{A}, the chamber (100, 200, 300, 400, 500, 600, 700, 800) may be buoyant with respect to, and float when placed in, a liquid such as a culture medium 130, water, a water-based nutrient bath, etc. In representative chamber (100, 200, 300, 400, 500, 600, 700, 800) embodiments, the chamber (100, 200, 300, 400, 500, 600, 700, 800) or its component second layer 105 and/or first layer 110 may comprise a material which facilitates such buoyancy. Stated another way, representative chamber (100, 200, 300, 400, 500, 600, 700, 800) embodiments, the chamber (100, 200, 300, 400, 500, 600, 700, 800) may have a selected density or selected shape, or its component second layer 105 and/or first layer 110 may each comprise materials having a density (or combined density) or shape, which facilitates such buoyancy. For example and without limitation, the first layer 110 may comprise a comparatively thin layer of glass (in the depth or thickness (z) dimension), the second layer 105 may comprise a less dense (or more buoyant) material and/or a comparatively hydrophobic material (such as polystyrene), and may also be comparatively thicker than the first layer 110, resulting in the chamber (100, 200, 300, 400, 500, 600, 700, 800) as a whole being comparatively less dense than the culture medium 130 and hence buoyant in a culture medium 130, as illustrated for chamber 100_{A} in FIGS. 7 and 8. Also for example, the second layer 105 may be comprised of an inert closed-cell polymeric foam (such as a styrofoam). Alternatively, one or both of the second layer 105 and/or first layer 110 may have a structure, configuration and/or shape which facilitates buoyancy, such as a rim or lip (165, 165_{A}) illustrated in FIGS. 12 and 13. For example and without limitation, although the density of polystyrene is about 1.05 gm/cm³, a representative chamber (100, 200, 300, 400, 500, 600, 700, 800) may float because the specific gravity is sufficiently close to 1, the chambers are light enough, and one or more of the materials comprising the chamber repels water, such as a second layer 105 comprising polystyrene, for example and without limitation. The generally planar lateral configuration (in the x, y dimensions) and relatively large surface area per volume of the chamber (100, 200, 300, 400, 500, 600, 700, 800), also contributes to the ability of the chambers to float. The surface area of the exposed side of the first layer 110 may be between 170 square mm and 1000 square mm, and particularly between 400 and 900 square millimeters, for example and without limitation.

As a result, two representative chamber (100, 200, 300, 400, 500, 600, 700, 800) embodiments may be arranged in a cell culture sandwich configuration, as illustrated, with one chamber (100, 200, 300, 400, 500, 600, 700, 800), illustrated as chamber 100_{A}, floating in the culture medium 130 above and without contacting the non-floating chamber (100, 200, 300, 400, 500, 600, 700, 800) which is submerged in the culture medium 130, illustrated as chamber 100_{B}. Typically each chamber is growing a different cell culture, such as neurons growing in the well 150 of chamber 100_{A} and a glial feeder layer growing in well 150 of chamber 100_{B}, respectively cell cultures 125_{A} and 125_{B}. As mentioned above, this allows the neurons to benefit from the trophic factors released from the glia. Significantly, no “feet” need to be included in the chambers (100, 200, 300, 400, 500, 600, 700, 800), and no “feet” need to be removed to allow imaging of the cell cultures in the wells 150 of the two representative chamber (100, 200, 300, 400, 500, 600, 700, 800) embodiments. For single layer cell cultures 125, either a buoyant or a non-buoyant chamber (100, 200, 300, 400, 500, 600, 700, 800) may be utilized.

FIGS. 7 and 8 also illustrate that different representative chamber (100, 200, 300, 400, 500, 600, 700, 800) embodiments may be utilized together, for example, chambers (100, 200, 300, 400, 500, 600, 700, 800) comprising different materials in their respective second layers 105 and/or first layers 110, or different shapes, resulting in one chamber (chamber 100_{A}) which is buoyant within the culture medium 130 and another chamber (chamber 100_{B}) which is not buoyant but is submersible within the culture medium 130.

For example and without limitation, in a representative embodiment, a representative chamber 100_{A} embodiment is generally circular and has a diameter of about 25 mm, and having a well 150 which has a circular shape in the lateral dimension, with a diameter of about 21 mm. For this representative chamber 100_{A} embodiment, the second layer 105 is annular, having an annular width of about 1-2 mm surrounding the well 150.

For example, the cells for chamber 100_{A} and chamber 100_{B} are each plated on a respective region 116 of the surface 112 of the first layer 110 of the chamber 100, e.g., comprising glass (which may be pre-treated with a cellular adhesive agent, such as poly-D-lysine, for example and without limitation). For culturing, the chamber 100_{B} is placed upside up in a standard 35 mm Petri dish filled with a culture medium 130 (with those cells facing upwards), and being non-buoyant, is submerged in the culture medium 130, followed by the chamber 100_{A} being placed upside down and over the chamber 100_{B} (with the cells of chamber 100_{A} facing the bottom of the Petri dish 120, as illustrated), and being buoyant, is only partially submerged and floats within or on the culture medium 130.

The Petri dish 120 with the floating chamber 100_{A} above the submerged chamber 100_{B} may be placed in an incubator. After a desired time in vitro, each chamber 100_{A} and chamber 100_{B} may be removed, positioned right side up, and used for experiments. When each of the chambers (100, 200, 300, 400, 500, 600, 700, 800) with a cell culture 125 is removed from the Petri dish 120, due to surface adhesion forces, the culture medium 130 stays in the well 150 even if the chamber (100, 200, 300, 400, 500, 600, 700, 800) is inverted or upside down (i.e., the culture medium 130 typically may only be removed by aspiration or vigorous shaking (with the latter typically not recommended)). Typically, the volume of the culture medium 130 in the well 150 is about 40 μl, but may be reduced to 10 μl if necessary.

This floating sandwich configuration of two representative chamber (100, 200, 300, 400, 500, 600, 700, 800) embodiments provides several advantages. It greatly facilitates preparation of sandwich co-cultures. It facilitates handling, as the chambers (100, 200, 300, 400, 500, 600, 700, 800) can be very easily manipulated and removed using forceps. It minimizes the possibility of unintended cytotoxicity, since the possibility of any of the cell cultures 125 having contact with air is reduced.

Representative chamber (100, 200, 300, 400, 500, 600, 700, 800) embodiments also may be advantageous when used in experiments involving microscopic observations using DIC and fluorescence optics. Low density neuronal cultures are preferred because single neurons can be studied without other cells in the background. To avoid exposing the cells to air and thus potentially compromising cell viability, the instant representative chamber (100, 200, 300, 400, 500, 600, 700, 800) embodiments eliminates the need to remove a plated coverslip from a Petri dish, and consequently neurons of a cell culture 125 may be always remain submerged in culture medium 130, including during imaging and experimentation.

A unique feature of a representative chamber (100, 200, 300, 400, 500, 600, 700, 800) embodiment is that it may float (e.g., with the neurons growing upside down). The flotation allows for significant improvements in the method of culturing the neurons. Because a chamber (100, 200, 300, 400, 500, 600, 700, 800) can be fabricated to be buoyant, the buoyant embodiments are very well suited to grow neuro-glial sandwich co-cultures. Additionally, the neurons growing in the chambers (100, 200, 300, 400, 500, 600, 700, 800) are immediately ready for many types of experiments, such as electrophysiology, ion imaging, immunocytochemistry, cell viability, transfection and others.

In addition, the well 150, after it is positioned upside up, becomes a superfusion-ready chamber. To facilitate superfusion, the well 150 having an oval, elliptical or rhomboid shape, for example, and also there may be optional recesses 115 (or cutouts) made in the well 150 for the use of inflow and outflow devices. FIG. 9 is an isometric view of a representative second chamber 200 embodiment. The second chamber 200 embodiment illustrates this additional feature available for any of the various representative chamber (100, 200, 300, 400, 500, 600, 700, 800) embodiments, namely, the elliptical or rhomboid shape of the well 150_{A} and/or the addition of an optional recess 115 in the well 150 (of the second layer 105, illustrated as second layer 105_{A}), illustrated in FIG. 9 with two recesses 115 spaced apart from each other, typically arranged directly opposite each other on opposite or opposing sides of the well 150_{A}, illustrated at opposite first and second ends 132, 134. While illustrated with two recesses 115 in FIG. 9, those having skill in the art will recognize that more or fewer recesses 115 may be utilized equivalently and all such variations are within the scope of the disclosure. FIG. 9 also illustrates a well 150_{A} having an oval or elliptical shape, such as to facilitate laminar flow, with an inflow of a liquid in one of the recesses 115, and with the liquid outflow aspirated from the other recess 115.

Continuing to refer to FIG. 9, as mentioned above, the well 150 may have any shape and size, such as circular, triangular, square, rectangular, polygonal, or may be shaped to provide for laminar flow during superfusion, such as oval (ovoid or elliptical), rhomboid, a rhombus, a parallelogram, or another shape suitable for allowing generally laminar flow. As illustrated in FIG. 9, the well 150A may have a first end 132 and a second end 134, the well further having a laminar flow shape extending laterally between the first and second ends 132, 134, illustrated as elliptical or oval, for example and without limitation. Also for example, the laminar flow shape may selected from the group consisting of: elliptical, oval, parallelogram, rhombus, rhomboid, and combinations thereof.

FIG. 10 is an isometric view of a representative third chamber 300 embodiment. The third chamber 300 embodiment illustrates another feature available for any of the various representative chamber (100, 200, 300, 400, 500, 600, 700, 800) embodiments, in addition to the use of recesses 115 in the wells 150, namely, the use of a plurality of wells 150 in a chamber 300, illustrated as wells 150_{B} and 150_{C} (of the second layer 105, illustrated as second layer 105_{B}), which are in (fluid) communication or connection with each other using a canal 140 (e.g., formed as a half-tube or half-pipe, having any type of cross-section (e.g., half-round, half-square, etc.)), also recessed in the second layer 105, generally at about the same depth as the wells 150), and with each of the wells 150_{B} and 150_{C} illustrated as having a single recess 115, arranged opposite each other on the respective wells 150. FIG. 10 also illustrates wells 150_{B} and 150_{C} respectively having rhomboidal and rhombus shapes (or diamond-shaped, equivalently), and also having slightly different sizes. This third chamber 300 embodiment is also superfusion-ready, also using inflow and outflow devices, such as manifold tubes or syringes, or may be constructed in other ways. A particular advantage of this multi-well configuration of the third chamber 300 embodiment is for the study of cellular movement, in which different cell types are plated in each of the wells 150_{B} and 150_{C}, with the cells of the cell cultures 125 able to migrate (e.g., along interconnecting canal 140 between wells 150 and possibly to another well 150), such as based upon the release of chemo-attractant molecules from one of the cell cultures 125. The length of the canal 140 may have any suitable dimension, including zero, when the wells 150_{B} and 150_{C} simply open into each other, or longer, e.g., 0 mm to 30 mm, and any dimension in between, depending on the selected experiment to be performed, the overall size of the representative chamber (100, 200, 300, 400, 500, 600, 700, 800), and the overall sizes of the wells 150.

FIG. 11 is a cross-sectional view (through the D-D′ plane and through the E-E′ plane) of the representative second and/or third chamber 200, 300 embodiments illustrated in FIGS. 9 and 10, further arranged with influx and aspiration tubes 220, 225 for superfusion of cell cultures. The well 150 may have at least one recess 115 along a perimeter of the well 150. The at least one recess 115 may be configured to accommodate the tip (224) of an aspiration or outflow tube 220 or the tip (222) of an influx or inflow tube (225). In a representative chamber 200 embodiment, the well 150 may have two recesses 115, while in another representative chamber 300 embodiment, each of the wells 150_{A} and 150_{B} illustrated as having a single recess 115. One recess 115 may be configured to accommodate the tip of an aspiration or outflow tube, and one recess 115 may be configured to accommodate the tip of an influx or inflow tube.

Also as illustrated in FIG. 11, a recess 115 may have a different depth than another recess 115, illustrated as one of the recess 115 having a reduced thickness, leaving a small shelf or tab 160 in the recess 115. This is particularly useful for use of the tip (224) of an aspiration or outflow tube 220, which is prevented from reaching the bottom of the well 150, helping to assure that excessive amounts of fluid or other liquid are not removed from the well 150, which could leave an insufficient amount of fluid or liquid to bathe the cell culture 125.

FIG. 12 is an isometric view of a representative fourth chamber 400 embodiment. FIG. 13 is a cross-sectional view (through the F-F′ plane) of the representative fourth chamber 400 embodiment illustrated in FIG. 12. The representative fourth chamber 400 embodiment differs from the other illustrated chamber embodiments, with the second layer 105 (illustrated as second layer 105_{C}) further comprising a raised rim (or lip) 165 arranged about the periphery or circumference of the second layer 105_{C}, as illustrated. Alternatively, when a chamber 400 is to be utilized upside down, a first layer 110 may instead have a raised rim (or lip) 165_{A} arranged about the periphery or circumference of the first layer 110, illustrated using dashed lines in FIGS. 12 and 13. The representative fourth chamber 400 embodiment is an example in which one or both of the second layer 105 and/or first layer 110, and/or the chamber as a whole, may have a structure, configuration and/or shape which facilitates buoyancy, independently of the density of the material composition of the first and/or first layers 105, 110, such as a structural rim or lip (165, 165_{A}), as illustrated in FIGS. 12 and 13.

FIG. 14 is an isometric view of a representative fifth chamber 500 embodiment. FIG. 15 is a cross-sectional view (through the G-G′ plane) of the representative fifth chamber 500 embodiment illustrated in FIG. 14. The representative fifth chamber 500 embodiment differs from the other representative chambers (100, 200, 300, 400, 600, 700, 800) insofar as it is not a laminate structure, and instead the functionality of the second layer 105 and first layer 110 has been combined into a single, integrally-formed second layer 105_{D}, as illustrated. For example, the second layer 105_{D} may be comprised of a glass or comparatively clear or otherwise optically transmission polymer, in which well 150 is fabricated (e.g., molded, ground, drilled, and/or polished). Depending upon the selected material forming the second layer 105_{D}, the resulting fifth chamber 500 may or may not be buoyant.

FIG. 14 also illustrates a well 150_{D} having an oval or elliptical shape, such as to facilitate laminar flow, without the additional use of any recess(es) 115. Such a well 150_{D}, having an oval or elliptical shape in the lateral dimension, may be utilized in any of the various representative chamber (100, 200, 300, 400, 500, 600, 700, 800) embodiments.

FIG. 16 is an isometric view of a representative sixth chamber 600 embodiment. FIG. 17 is a cross-sectional view (through the H-H′ plane) of the representative sixth chamber 600 embodiment illustrated in FIG. 16. The representative sixth chamber 600 embodiment differs from the other representative chambers (100, 200, 300, 400, 700, 800) insofar as the first layer 110_{A} has a smaller lateral dimension than the second layer 105_{E}, as illustrated, rather than having the same or similar lateral dimensions. The first layer 110_{A} also includes a micro-grid 175 (comprising generally two sets of spaced-apart lines arranged orthogonally to each other to form a grid 175) extending at least throughout the respective region 116 of the surface 112 of the first layer 110A of the chamber 600, such as to aid in measurement and/or localization of the various cells of the cell culture 125. The micro-grid 175 may be marked as indicia, or added (e.g., ground or scribed) into the first layer 110_{A}, or arranged or integrally-formed with the first layer 110_{A}, for example. Also for example, the micro-grid 175 may also include other indicia useful for measurements, such as numberings to coincide with each line of the micro-grid 175, and may provide any selected resolution or line spacing.

FIG. 16 also illustrates a well 150_{E} having a rhomboid, rhombus or diamond shape, as an example of a type of laminar flow shape to facilitate laminar flow, without the additional use of any recess(es) 115. Such a well 150_{E}, having a rhomboid, rhombus or diamond shape in the lateral dimension may be utilized in any of the various representative chamber (100, 200, 300, 400, 500, 600, 700, 800) embodiments.

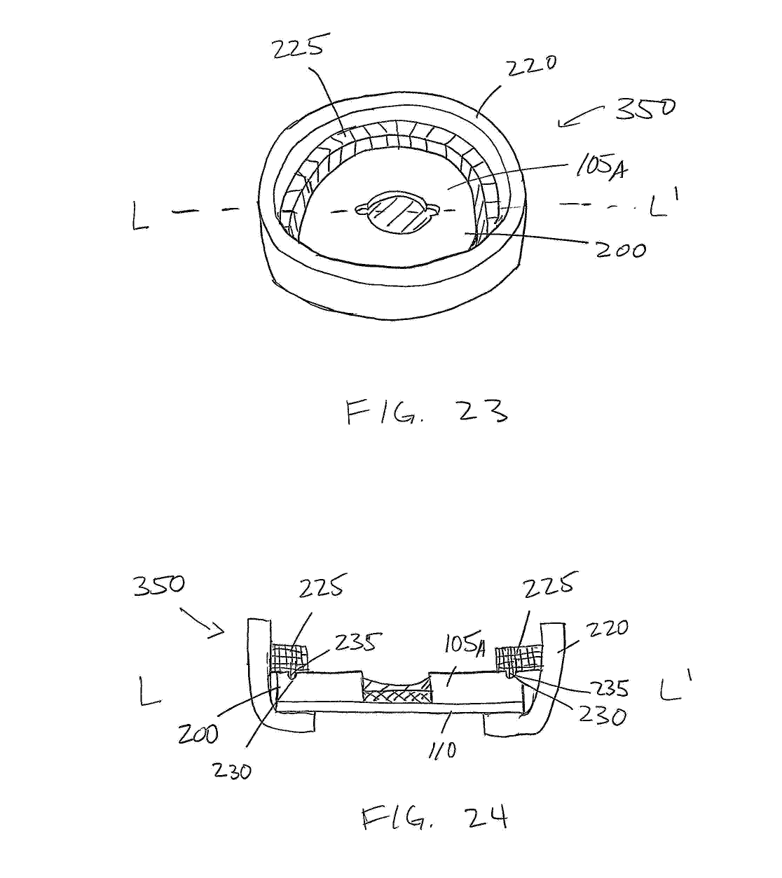

FIG. 18 is an isometric view of a representative seventh chamber 700 embodiment showing a representative alignment detent 170, alignment recesses 180, 185, and an alignment indicia (or marking) 190, which may be provided in either the second layer 105_{F} (e.g., alignment indicia (or marking) 190 on the upper surface of the second layer 105_{F}) or either or both the first layer 110 and the second layer 105_{F} (e.g., alignment detent 170, alignment recesses 180, 185), for use in any representative chamber embodiment, for example, to provide a mechanism to repeatedly align the chamber 700 in the same orientation during imaging. Any of these alignment detents 170, alignment recesses 180, 185, and/or an alignment indicia (or marking) 190 may be included as an option in any of the representative chambers (100, 200, 300, 400, 500, 600, 700, 800), and may utilized separately or individually or in any combination with each other, for example and without limitation. FIG. 18 also illustrates a groove 230 recessed into the second layer 105_{F}, which may be utilized to mate or match with a sealing gasket 225 discussed below with reference to FIGS. 23 and 24.

FIG. 19 is an isometric view of a representative eighth chamber 800 embodiment. FIG. 20 is a cross-sectional view (through the J-J′ plane) of the representative eighth chamber 800 embodiment illustrated in FIG. 19. The representative eighth chamber 800 embodiment differs from the other representative chambers (100, 200, 300, 400, 500, 600 700) insofar as the first layer 110_{B} has a greater lateral dimension than the second layer 105 (illustrated as a second layer 105A), rather than having the same or similar lateral dimensions. This feature may be useful, for example, for holding the eighth chamber 800 in various types of chamber holder systems.

FIG. 21 is an isometric view of a representative first chamber holder system 250 embodiment. FIG. 22 is a cross-sectional view (through the K-K′ plane) of the representative first chamber holder system 250 embodiment illustrated in FIG. 21. For purposes of explanation, the representative first chamber holder system 250 is illustrated using an eighth chamber 800 embodiment, although any of the other representative chambers (100, 200, 300, 400, 500, 600, 700) may also be used equivalently. The representative first chamber holder system 250 is adapted to be used with a light microscope. For example, the representative first chamber holder system 250 may have various shapes provided that the annular portion of the holder is about 35 mm in diameter and fits the stages of common microscopes (Zeiss, Leica, Olympus, etc.). The representative first chamber holder system 250 comprises a first, top holder 205 and a second, bottom holder 210. The first holder 205 also includes one or more flexible, compressible sealing gaskets 215 that serve to clamp and seal the representative chamber (100, 200, 300, 400, 500, 600, 700, 800) within the representative first chamber holder system 250. Typically, the first holder 205 and second holder 210 may be fitted into each other, securing the representative chamber (100, 200, 300, 400, 500, 600, 700, 800) between the first and second holders 205, 210, and all are held together by compression and frictional forces.

FIG. 23 is an isometric view of a representative second chamber holder system 350 embodiment. FIG. 24 is a cross-sectional view (through the L-L′ plane) of the representative second chamber holder system 350 embodiment illustrated in FIG. 23. The representative second chamber holder system 350 comprises a holder 220 and a flexible, deformable sealing gasket 225 around the interior of the holder 220, which secures the representative chamber (100, 200, 300, 400, 500, 600, 700, 800) within the interior of the holder 220, and which seals the representative second chamber holder system 350.

The first or second chamber holder system 250, 350 are typically annular or open at least to some degree in their respective interiors, to allow for light microscopy of the representative chamber (100, 200, 300, 400, 500, 600, 700, 800) held in the first or second chamber holder system 250, 350. To conduct superfusion, the representative chamber (100, 200, 300, 400, 500, 600, 700, 800) is secured in a first or second chamber holder system 250, 350 that fits for microscope stages. The representative chamber (100, 200, 300, 400, 500, 600, 700, 800) may be secured in a first or second chamber holder system 250, 350 using a sealing gasket 215, 225. The first or second chamber holder system 250, 350 may have various shapes provided that the annular portion of the first or second chamber holder system 250, 350 is about 35 mm in diameter and fits the stages of common microscope (Zeiss, Leica, Olympus, etc.), for example and without limitation.

In various embodiments, the first or second chamber holder system 250, 350 may also interlock with the representative chamber (100, 200, 300, 400, 500, 600, 700, 800). The first or second chamber holder system 250, 350 may also incorporate ports for the inflow and outflow to allow quick connection of inflow and outflow lines for superfusion. In various embodiments, an annular gasket 225 (e.g., comprising silicone) may be used but it may be advantageous to have a gasket with a different cross section to have minimal sealing pressure. In one embodiment, the representative chamber (100, 200, 300, 400, 500, 600, 700, 800) is provided with a groove 230 (illustrated in FIG. 18), and the annular gasket 225 has a mating or matching raised ring 235 portion. In another embodiment not separately illustrated, the first or second chamber holder system 250, 350 has springs to provide sealing pressure. In various embodiments, vacuum grease may also be used to provide sealing pressure.

The superfusion may be performed directly in the representative chamber (100, 200, 300, 400, 500, 600, 700, 800), without the need to remove the cells. For experiments that involve superfusion, such as electrophysiology or ionic transients, the shape of the well 150 may be optimized for laminar flow. To achieve laminar flow, the entry and exit of the solution being used may expand gradually. To achieve laminar flow the entry and exit of solution is designed to expand and contract gradually, usually in two dimensions as illustrated in various Figures, but also in a third dimension, like a rectangular funnel. For other experiments (immunocytochemistry, cell viability etc.) the well 150 may be circular, also as illustrated in various Figures. The outflow recess 115 may accommodate an aspiration tube constructed such that the cells do not dry out if buffer supply is accidentally interrupted.

Performing superfusion directly in the representative chamber (100, 200, 300, 400, 500, 600, 700, 800) allows the cells to be kept submerged in culture medium 130 and not be exposed to air. It also allows the temperature to be controlled without the need to heat or cool the entire representative chamber (100, 200, 300, 400, 500, 600, 700, 800). The generally comparatively small volume of the well 150 allows the temperature to be controlled by controlling the temperature of the inflowing buffer. Superfusion may be performed by inserting an aspiration tube in one of the recesses 115, and inserting an influx tube in another recess 115. Thus, by causing liquid to flow directly over the cells within the well 150, superfusion is much easier than with traditional methods.

A further advantage of the comparatively small size of the well 150 in the representative chamber (100, 200, 300, 400, 500, 600, 700, 800) is that it may be useful for culturing cells that are only available in limited quantities. Further, manipulations involved in the maintenance of cultures are minimal because the overall volume of the medium in the Petri dish 120 (about 2 ml) is very large compared to the volume of the cells in the well. Sterile water only needs to be added to compensate for evaporation, and the culture medium 130 does not need to be exchanged.

The comparatively small volume of the well 150 also facilitates plasmid-mediated cell transfections. Transfection are performed when the representative chamber (100, 200, 300, 400, 500, 600, 700, 800) is upside up and the volume can be reduced below 50 μ. After the transfections, the representative chamber (100, 200, 300, 400, 500, 600, 700, 800) is turned upside down, placed back into a Petri dish 120 filled with culture medium 130 and returned to and incubator.

The comparatively small volume of the well 150 may reduce the costs of experiments, especially those requiring expensive agents and/or antibodies, as smaller amounts will be used. Due to the comparatively small volume of the well 150, it is also very easy to transfect the neurons with plasmids. To this end, the standard lipofectamine 2000 protocol can be used.

After fixing the cells with, for example, 4% paraformaldehyde, and conducting an immunocytochemical staining, the chambers can be stored stacked one on top of another. This feature saves space. In one embodiment, up to eight chambers may be stored stacked within a standard Petri dish 120.

The representative chamber (100, 200, 300, 400, 500, 600, 700, 800) also may be sterilized and cleaned. The portion of the first layer 110 that forms part of the well 150 of the representative chamber (100, 200, 300, 400, 500, 600, 700, 800) may be coated to ensure adhesion of cells or neurons to the surface. One of laminin, poly-D-lysine, collagen, fibronectin, polyethylenimine may be used to coat the portion of the first layer 110 that forms part of the well 150 of the representative chamber (100, 200, 300, 400, 500, 600, 700, 800), to promote adhesion of the cells to the surface.

When using sandwich neuro-glial co-cultures, the disclosed representative chambers (100, 200, 300, 400, 500, 600, 700, 800) solve many of the problems of the existing methods. The neurons can float upside down and benefit from the trophic factors released by glia growing underneath. No paraffin feet are needed. When the representative chamber (100, 200, 300, 400, 500, 600, 700, 800) is removed from the Petri dish 120, there is always culture medium 130 in the well 150, as this culture medium 130 would require aspiration to be removed, due to the hydrostatic forces mentioned above. A representative chamber (100, 200, 300, 400, 500, 600, 700, 800) is secured in a first or second chamber holder system 250, 350 with two or more connected wells 150 also may be designed to culture two or more types of cells at the same level, as illustrated in FIGS. 9 and 10. This may be useful to study projecting neurons (such as dopaminergic neurons) innervating their target neurons (such as neurons from basal ganglia).

The representative chambers (100, 200, 300, 400, 500, 600, 700, 800) is secured in a first or second chamber holder system 250, 350 may also be sized and shaped to fit any commercially available holder, including those designed to fit existing glass coverslips, e.g., accommodating a thickness or depth of the first layer 110 (e.g., first layer 110B) having a thickness (or depth) on the order of 0.1 mm to 0.3 mm, for example and without limitation. The representative first or second chamber holder systems 250, 350 also may be sized and shaped to accommodate any of the various representative chambers (100, 200, 300, 400, 500, 600, 700, 800), e.g., having a thickness (or depth) on the order of 0.4 mm to 1.0 mm, and may have integrated ports for medium inflow and outflow.

As mentioned above, the second layer 105 is comprised of any suitable material, such as a biocompatible or inert polymer or plastic, such as polystyrene or polytetrafluoroethylene (PTFE or Teflon), any type of biocompatible or inert glass, any type of biocompatible or inert polymeric foam, generally as a closed cell foam, or any type of biocompatible or inert metal or alloy, for example and without limitation. Other representative examples of biocompatible or inert polymers include, but are not limited to, fluorinated polymers or copolymers such as poly(vinylidene fluoride), poly(vinylidene fluoride-co-hexafluoropropene), poly(tetrafluoroethylene), and expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-co-trimethylene carbonate); poly(phosphoesters); poly(phosphoester urethane); poly(trimethylene carbonate); poly(iminocarbonate); poly(ethylene); and any derivatives, analogs, homologues, congeners, salts, copolymers and combinations thereof.

The biocompatible or inert polymers may also include, but are not limited to, poly(propylene) co-poly(ether-esters) such as, for example, poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers such as poly(vinyl chloride); poly(vinyl ethers) such as, for example, poly(vinyl methyl ether); poly(vinylidene halides) such as, for example, poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins such as poly(ethylene-co-vinyl alcohol) (EVAL), copolymers of acrylonitrile-styrene, ABS resins, and copolymers of ethylene-vinyl acetate; and any derivatives, analogs, homologues, congeners, salts, copolymers and combinations thereof. For example, an Aclar® PVC film may be utilized.

The biocompatible or inert polymers may further include, but are not limited to, polyoleins (such as Thermanox®), or poly(amides) such as Nylon 66 and poly(caprolactam); alkyd resins; poly(carbonates); poly(oxymethylenes); poly(imides); poly(ester amides); poly(ethers) including poly(alkylene glycols) such as, for example, poly(ethylene glycol) and poly(propylene glycol); epoxy resins; polyurethanes; rayon; rayon-triacetate; biomolecules such as, for example, fibrin, fibrinogen, starch, poly(amino acids); peptides, proteins, gelatin, chondroitin sulfate, dermatan sulfate (a copolymer of D-glucuronic acid or L-iduronic acid and N-acetyl-D-galactosamine), collagen, hyaluronic acid, and glycosaminoglycans; other polysaccharides such as, for example, poly(N-acetylglucosamine), chitin, chitosan, cellulose, cellulose acetate, cellulose butyrate, cellulose acetate butyrate, cellophane, cellulose nitrate, cellulose propionate, cellulose ethers, and carboxymethylcellulose; and any derivatives, analogs, homologues, congeners, salts, copolymers and combinations thereof. At least one of polymers can be a poly(ester amide), a poly(lactide) or a poly(lactide-co-glycolide) copolymer; and any derivatives, analogs, homologues, congeners, salts, copolymers and combinations thereof.

As mentioned above, the first layer 110 is comprised of any substantially optically transmissive material for a selected range of imaging wavelengths, such as a biocompatible or inert glass or polymer, such as a borosilicate glass or polystyrene latex, including the various polymers described above. Representative types of biocompatible or inert glasses include, in addition to borosilicate glass (any silicate glass having at least 5% of boric oxide): soda-lime glass, a lead glass (including a lead-alkali glass), aluminosilicate glass (having aluminum oxide in its composition), ninety-six percent silica glass, and fused silica glass.

As mentioned above, any type of adhesive 145 (also generally biocompatible or inert) may be utilized to bond the second layer 105 and the first layer 110 to form a cell culturing chamber (100, 200, 300, 400, 500, 600, 700, 800), including without limitation epoxies, cyanoacrylates, polyurethanes and other urethane and acrylic adhesives, polyimides, and in any form (such as paste, liquid, film, pellets, tape), or type (e.g. hot melt, reactive hot melt, thermosetting, pressure sensitive, contact), and with any type of cure (e.g., ultraviolet, infrared, heat).

The culture adhesive may be any substance or substances that facilitate(s) cell attachment to a material of the first layer 110. Examples include poly-lysine, poly-ornithine, collagen, laminin, matrigel, and a combination thereof. The culture adhesive may be applied at any suitable concentration. The culture adhesive may be diluted to a desired concentration prior to use.

The present disclosure is to be considered as an exemplification of the principles of the invention and is not intended to limit the invention to the specific embodiments illustrated. In this respect, it is to be understood that the invention is not limited in its application to the details of construction and to the arrangements of components set forth above and below, illustrated in the drawings, or as described in the examples. Systems, methods and apparatuses consistent with the present invention are capable of other embodiments and of being practiced and carried out in various ways.

Although the invention has been described with respect to specific embodiments thereof, these embodiments are merely illustrative and not restrictive of the invention. In the description herein, numerous specific details are provided, such as examples of electronic components, electronic and structural connections, materials, and structural variations, to provide a thorough understanding of embodiments of the present invention. One skilled in the relevant art will recognize, however, that an embodiment of the invention can be practiced without one or more of the specific details, or with other apparatus, systems, assemblies, components, materials, parts, etc. In other instances, well-known structures, materials, or operations are not specifically shown or described in detail to avoid obscuring aspects of embodiments of the present invention. In addition, the various Figures are not drawn to scale and should not be regarded as limiting.

Reference throughout this specification to “one embodiment”, “an embodiment”, or a specific “embodiment” means that a particular feature, structure, or characteristic described in connection with the embodiment is included in at least one embodiment of the present invention and not necessarily in all embodiments, and further, are not necessarily referring to the same embodiment. Furthermore, the particular features, structures, or characteristics of any specific embodiment of the present invention may be combined in any suitable manner and in any suitable combination with one or more other embodiments, including the use of selected features without corresponding use of other features. In addition, many modifications may be made to adapt a particular application, situation or material to the essential scope and spirit of the present invention. It is to be understood that other variations and modifications of the embodiments of the present invention described and illustrated herein are possible in light of the teachings herein and are to be considered part of the spirit and scope of the present invention.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. In addition, every intervening sub-range within range is contemplated, in any combination, and is within the scope of the disclosure. For example, for the range of 5-10, the sub-ranges 5-6, 5-7, 5-8, 5-9, 6-7, 6-8, 6-9, 6-10, 7-8, 7-9, 7-10, 8-9, 8-10, and 9-10 are contemplated and within the scope of the disclosed range.

It will also be appreciated that one or more of the elements depicted in the Figures can also be implemented in a more separate or integrated manner, or even removed or rendered inoperable in certain cases, as may be useful in accordance with a particular application. Integrally formed combinations of components are also within the scope of the invention, particularly for embodiments in which a separation or combination of discrete components is unclear or indiscernible. In addition, use of the term “coupled” herein, including in its various forms such as “coupling” or “couplable”, means and includes any direct or indirect electrical, structural or magnetic coupling, connection or attachment, or adaptation or capability for such a direct or indirect electrical, structural or magnetic coupling, connection or attachment, including integrally formed components and components which are coupled via or through another component.

Furthermore, any signal arrows in the drawings/Figures should be considered only exemplary, and not limiting, unless otherwise specifically noted. Combinations of components of steps will also be considered within the scope of the present invention, particularly where the ability to separate or combine is unclear or foreseeable. The disjunctive term “or”, as used herein and throughout the claims that follow, is generally intended to mean “and/or”, having both conjunctive and disjunctive meanings (and is not confined to an “exclusive or” meaning), unless otherwise indicated. As used in the description herein and throughout the claims that follow, “a”, “an”, and “the” include plural references unless the context clearly dictates otherwise. Also as used in the description herein and throughout the claims that follow, the meaning of “in” includes “in” and “on” unless the context clearly dictates otherwise.

The foregoing description of illustrated embodiments of the present invention, including what is described in the summary or in the abstract, is not intended to be exhaustive or to limit the invention to the precise forms disclosed herein. From the foregoing, it will be observed that numerous variations, modifications and substitutions are intended and may be effected without departing from the spirit and scope of the novel concept of the invention. It is to be understood that no limitation with respect to the specific methods and apparatus illustrated herein is intended or should be inferred. It is, of course, intended to cover by the appended claims all such modifications as fall within the scope of the claims.

It is claimed:

1. A chamber for a cell culture, the chamber insertable into a dish having a culture medium, the chamber comprising:
a first layer; and
a second layer coupled to the first layer to form a laminate structure, the second layer further comprising a well having at least one side wall, the well extending through the second layer, the well further having a first end and a second end, the well further having a laminar flow shape extending laterally between the first and second ends; and
wherein a predetermined portion of the first layer is substantially optically transmissive and is exposed in and forms a lower side of the well.

2. The chamber of claim 1, wherein the predetermined portion of the first layer further comprises:
a micro-grid.

3. The chamber of claim 1, wherein the second layer further comprises:
a plurality of recesses in the at least one side wall, a first recess of the plurality of recesses arranged at the first end and having a size adapted to accommodate a tip of an inflow device, and a second recess of the plurality of recesses spaced apart from and arranged at the second end opposite the first recess, the second recess having a size adapted to accommodate a tip of an outflow device.

4. The chamber of claim 1, wherein the well has a volume with a range of 10 to 50 microliters, and wherein the laminar flow shape is selected from the group consisting of: elliptical, oval, parallelogram, rhombus, rhomboid, and combinations thereof.

5. The chamber of claim 1, wherein the second layer further comprises:
a plurality of wells, the plurality of wells are coplanar with each other, each well having at least one side wall, each well extending through the second layer, and wherein a plurality of predetermined portions of the first layer are substantially optically transmissive and are exposed in and form corresponding lower sides of the plurality of wells; and
at least one channel coupling a first well of the plurality of wells to a second well of the plurality of wells.

6. The chamber of claim 1, wherein the second layer further comprises:
at least one alignment recess, alignment detent, or alignment indicia.

7. The chamber of claim 1, wherein the second layer further comprises:
a rim, the rim arranged around the circumference of the second layer.

8. The chamber of claim 1, wherein the second layer is comprised of a hydrophobic material or is comprised of a material having a first density less than a second density of a selected culture medium to provide that the chamber is buoyant in a selected culture medium.

9. The chamber of claim 1, wherein the well has a size adapted to retain culture medium within the well independently of a position of the chamber.

10. The chamber of claim 1, wherein the second layer comprises:
a biocompatible or inert closed-cell polymeric foam.

11. The chamber of claim 1, wherein the second layer further comprises:
a groove recessed into an upper surface of the second layer, and further comprising:
an annular chamber holder system, the annular chamber holder system comprising:
a first holder; and
a flexible gasket adapted to secure and seal the chamber against the first holder, wherein the flexible gasket further comprises a raised ring to mate with the groove of the second layer.

12. A chamber for a cell culture, the chamber insertable into a dish having a selected culture medium, the chamber buoyant in the selected culture medium, the chamber comprising:
a first layer comprising a substantially optically transmissive material; and
a second layer coupled to the first layer to form a laminate structure, the second layer further comprising a well having at least one side wall, the well extending through the second layer;
wherein a predetermined portion of the first layer is substantially optically transmissive and is exposed in and forms a lower side of the well; and
wherein the second layer is comprised of a hydrophobic material or is comprised of a material having a first density less than a second density of the selected culture medium.

13. The chamber of claim 12, wherein the second layer comprises:
a biocompatible or inert closed-cell polymeric foam.

14. The chamber of claim 12, wherein the well further comprises:
a first end and a second end, the well further having a laminar flow shape extending laterally between the first and second ends.

15. The chamber of claim 14, wherein the laminar flow shape is selected from the group consisting of: elliptical, oval, parallelogram, rhombus, rhomboid, and combinations thereof.

16. The chamber of claim 14, wherein the second layer further comprises:
a plurality of recesses in the at least one side wall, a first recess of the plurality of recesses arranged at the first end and having a size adapted to accommodate a tip of an inflow device, and a second recess of the plurality of recesses spaced apart from and arranged at the second end opposite the first recess, the second recess having a size adapted to accommodate a tip of an outflow device.

17. The chamber of claim 12, wherein the well has a size adapted to retain culture medium within the well independently of a position of the chamber.

18. The chamber of claim 12, wherein the second layer comprises a biocompatible or inert polymer or copolymer, selected from the group consisting of: fluorinated polymers or copolymers including poly(vinylidene fluoride), poly(vinylidene fluoride-co-hexafluoropropene), poly(tetrafluoroethylene), expanded poly(tetrafluoroethylene); poly(sulfone); poly(N-vinyl pyrrolidone); poly(aminocarbonates); poly(iminocarbonates); poly(anhydride-co-imides), poly(hydroxyvalerate); poly(L-lactic acid); poly(L-lactide); poly(caprolactones); poly(lactide-co-glycolide); poly(hydroxybutyrates); poly(hydroxybutyrate-co-valerate); poly(dioxanones); poly(orthoesters); poly(anhydrides); poly(glycolic acid); poly(glycolide); poly(D,L-lactic acid); poly(D,L-lactide); poly(glycolic acid-co-trimethylene carbonate); poly(phosphoesters); poly(phosphoester urethane); poly(trimethylene carbonate); poly(iminocarbonate); poly(ethylene); poly(propylene) co-poly(ether-esters), including poly(dioxanone) and poly(ethylene oxide)/poly(lactic acid); poly(anhydrides), poly(alkylene oxalates); poly(phosphazenes); poly(urethanes); silicones; silicone rubber; poly(esters); poly(olefins); copolymers of poly(isobutylene); copolymers of ethylene-alphaolefin; vinyl halide polymers and copolymers including poly(vinyl chloride); poly(vinyl ethers) including poly(vinyl methyl ether); poly(vinylidene halides) including poly(vinylidene chloride); poly(acrylonitrile); poly(vinyl ketones); poly(vinyl aromatics) such as poly(styrene); poly(vinyl esters) such as poly(vinyl acetate); copolymers of vinyl monomers and olefins including poly(ethylene-co-vinyl alcohol) (EVAL), copolymers of acrylonitrile-styrene, ABS resins, and copolymers of ethylene-vinyl acetate; poly(amides); poly(caprolactam); alkyd resins; poly(carbonates); poly(oxymethylenes); poly(imides); poly(ester amides); poly(ethers) including poly(alkylene glycols) including poly(ethylene glycol) and poly(propylene glycol); a poly(ester amide), a poly(lactide) or a poly(lactide-co-glycolide) copolymer; epoxy resins; polyurethanes; rayon; rayon-triacetate; biomolecules including fibrin, fibrinogen, starch, poly(amino acids); peptides, proteins, gelatin, chondroitin sulfate, dermatan sulfate (a copolymer of D-glucuronic acid or L-iduronic acid and N-acetyl-Dgalactosamine), collagen, hyaluronic acid, glycosaminoglycans; polysaccharides including poly(N-acetylglucosamine), chitin, chitosan, cellulose, cellulose acetate, cellulose butyrate, cellulose acetate butyrate, cellophane, cellulose nitrate, cellulose propionate, cellulose ethers, carboxymethylcellulose; and any derivatives, analogs, homologues, congeners, salts, copolymers and combinations thereof.

19. A plurality of chambers for sandwich cell cultures, the plurality of chambers insertable into a dish having a selected culture medium, the plurality of chambers comprising:
a first chamber; and
a second chamber;
each of the first and second chambers comprising:
a first layer; and
a second layer coupled to the first layer to form a laminate structure, the second layer further comprising a well having at least one side wall, the well extending through the second layer, and wherein a predetermined portion of the first layer is substantially optically transmissive and is exposed in and forms a lower side of the well;
wherein the second layer of the first chamber is comprised of a hydrophobic material or is comprised of a material having a first density less than a second density of the selected culture medium to provide that the first chamber is buoyant in the selected culture medium; and
wherein the second layer of the second chamber is comprised of a material having a third density greater than the second density of the selected culture medium to provide that the second chamber is submergible in the selected culture medium.

20. The plurality of chambers of claim 19, wherein at least one well further comprises:
a first end and a second end, the at least one well further having a laminar flow shape extending laterally between the first and second ends, wherein the laminar flow shape is selected from the group consisting of: elliptical, oval, parallelogram, rhombus, rhomboid, and combinations thereof.
